(19) United States
(12) Patent Application Publication
(10) Pub. No.: US 2020/0323803 A1
(43) Pub. Date: Oct. 15, 2020

(54) COMPOSITIONS AND METHODS FOR GLUCOSE CONTROL

(71) Applicant: LEADING BIOSCIENCES, INC., Carlsbad, CA (US)

(72) Inventors: John Rodenrys, Carlsbad, CA (US); Michael Dawson, Carlsbad, CA (US)

(21) Appl. No.: 16/756,302

(22) PCT Filed: Oct. 24, 2018
(86) PCT No.: PCT/US2018/057379

§ 371 Date: Apr. 15, 2020

Related U.S. Application Data
(60) Provisional application No. 62/576,479, filed on Oct. 24, 2017.

(51) Int. Cl. A61K 31/195; A61K 47/10; A61K 47/26; A61K 47/02; A61P 3/10

(52) U.S. Cl. CPC A61K 31/195; A61K 47/10; A61K 9/0053; A61K 47/02; A61P 3/10; A61K 47/26

(57) ABSTRACT

Disclosed herein are, inter alia, pharmaceutical compositions useful for treating diabetes, lowering blood glucose levels in diabetic patients, and improving glycemic control in diabetic patients.

### CROSS-REFERENCE TO RELATED APPLICATIONS

This application claims priority to U.S. Application No. 62/576,479 filed Oct. 24, 2017, the disclosure of which is incorporated herein by reference in its entirety.

### BACKGROUND

A high number of diabetic patients undergo surgical procedures when compared to non-diabetic patients. Surgical procedures require fasting and cessation of diabetes medications, and cause changes in glucose homeostasis and hyperglycemia, which are risks factors for endothelial dysfunction, postoperative sepsis, impaired wound healing, and cerebral ischemia. Diabetic crises (e.g., diabetic ketoacidosis, hyperglycemic hyperosmolar syndrome) can arise during surgery or postoperatively, with a negative prognosis for diabetic patients.

The disclosure is directed to pharmaceutical compositions that are useful in managing glucose in diabetic patients undergoing surgical procedures, as well as diabetic patients not undergoing surgical procedures or who are in non-hospital settings. Disclosed herein, inter alia, are solutions to these and other problems in the art.

### BRIEF SUMMARY

Provided herein are methods for lowering blood glucose levels in a diabetic patient by administering to the diabetic patient a therapeutically effective amount of a pharmaceutical composition comprising tranexamic acid. Provided herein are methods for treating diabetes in a diabetic patient by administering a therapeutically effective amount of a pharmaceutical composition comprising tranexamic acid. Provided herein are methods for improving glycemic control in a diabetic patient by administering a therapeutically effective amount of a pharmaceutical composition comprising tranexamic acid. In embodiments, the composition is orally administered as a liquid dosage form or a solid dosage form. In embodiments, the composition is administered (i) prior to a surgical procedure; (ii) after a surgical procedure; or (iii) prior to the surgical procedure and after the surgical procedure. In embodiments, the method for lowering blood glucose levels is a method for reducing post-surgical blood glucose levels. In embodiments, the composition is orally administered during the surgical procedure. In embodiments, the composition is administered to a diabetic patient who is not undergoing a surgical procedure or who is in a non-hospital setting. In embodiments, the composition is administered chronically. In embodiments, the composition further comprises polyethylene glycol, glucose, electrolytes, or a combination of two or more thereof. In embodiments, the tranexamic acid preserves and protects insulin receptors.

Provided herein are methods for lowering blood glucose levels in a diabetic patient by administering to the diabetic patient a therapeutically effective amount of a pharmaceutical composition comprising: (i) a protease inhibitor, (ii) polyethylene glycol, or (iii) a protease inhibitor and polyethylene glycol. Provided herein are methods for treating diabetes in a diabetic patient by administering a therapeutically effective amount of a pharmaceutical composition comprising: (i) a protease inhibitor, (ii) polyethylene glycol, or (iii) a protease inhibitor and polyethylene glycol. Provided herein are methods for improving glycemic control in a diabetic patient by administering a therapeutically effective amount of a pharmaceutical composition comprising: (i) a protease inhibitor, (ii) polyethylene glycol, or (iii) a protease inhibitor and polyethylene glycol. In embodiments, the composition is orally administered as a liquid dosage form or a solid dosage form. In embodiments, the composition is administered (a) prior to a surgical procedure; (b) after a surgical procedure; or (c) prior to the surgical procedure and after the surgical procedure. In embodiments, the method for lowering blood glucose levels is a method for reducing post-surgical blood glucose levels. In embodiments, the composition is orally administered during the surgical procedure. In embodiments, the composition is administered to a diabetic patient who is not undergoing a surgical procedure or who is in a non-hospital setting. In embodiments, the composition is administered chronically to a diabetic patient. In embodiments, the composition comprises a protease inhibitor and, optionally, one or more of polyethylene glycol, glucose, and electrolytes. In embodiments, the protease inhibitor is a serine protease inhibitor, a matrix metalloproteinase inhibitor, a lipase, or a combination of two or more thereof. In embodiments, the protease inhibitor is tranexamic acid, gabexate mesilate, camostat mesilate, nafamostat mesilate, α-1-anti-trypsin, α-2-macroglobulin, pancreatic lipase, gastric lipase, pancreatic lipase-related protein 1, pancreatic lipase-related protein 2, aprotinin, or a combination of two or more thereof. In embodiments, the composition comprises polyethylene glycol and, optionally, one or more of glucose and electrolytes. In embodiments, the protease inhibitor preserves and protects insulin receptors.

Provided herein are methods for lowering blood glucose levels in a diabetic patient by orally administering to the diabetic patient a therapeutically effective amount of a pharmaceutical composition comprising an insulin receptor protease inhibitor. Provided herein are methods for treating diabetes in a diabetic patient by administering a therapeutically effective amount of a pharmaceutical composition comprising an insulin receptor protease inhibitor. Provided herein are methods for improving glycemic control in a diabetic patient by administering a therapeutically effective amount of a pharmaceutical composition comprising an insulin receptor protease inhibitor. In embodiments, the composition is orally administered (a) prior to a surgical procedure; (b) after a surgical procedure; or (c) prior to the surgical procedure and after the surgical procedure. In embodiments, the method for lowering blood glucose levels is a method for reducing post-surgical blood glucose levels. In embodiments, the composition is orally administered during the surgical procedure. In embodiments, the composition is administered to a diabetic patient who is not undergoing a surgical procedure or who is in a non-hospital setting. In embodiments, the composition is administered chronically. In embodiments, the insulin receptor protease inhibitor is a serine protease inhibitor, a matrix metalloproteinase inhibitor, a lipase, or a combination of two or more thereof. In embodiments, the insulin receptor protease inhibitor is tranexamic acid, gabexate mesilate, camostat mesilate, nafamostat mesilate, α-1-anti-trypsin, α-2-macroglobulin, pancreatic lipase, gastric lipase, pancreatic lipase-related protein 1, pancreatic lipase-related protein 2, aprotinin, or a combination of two or more thereof. In embodiments, the composition further comprises polyethylene glycol, glucose, electrolytes, or a combination of two or more thereof. In embodiments, the protease inhibitor preserves and protects insulin receptors.

Provided herein are aqueous pharmaceutical compositions comprising about 1.0 wt % to about 1.2 wt % of tranexamic acid, about 4.0 wt % to about 4.4 wt % of polyethylene glycol having an average molecular weight of about 3,350, about 4.8 wt % to about 5.2 wt % of glucose, about 0.2 wt % to about 0.6 wt % of sodium sulfate, about 0.1 wt % to about 0.14 wt % of sodium bicarbonate, about 0.08 wt % to about 0.21 wt % of sodium chloride, and about 0.03 wt % to about 0.07 wt % of potassium chloride. In embodiments, the pharmaceutical compositions comprise about 1.1 wt % of tranexamic acid, about 4.2 wt % of polyethylene glycol having an average molecular weight of about 3,350, about 5.0 wt % of glucose, about 0.4 wt % of sodium sulfate, about 0.12 wt % of sodium bicarbonate, about 0.1 wt % of sodium chloride, and about 0.05 wt % of potassium chloride.

These and embodiments of the disclosure are provided in more detail herein.

### BRIEF DESCRIPTION OF THE DRAWINGS

With reference to FIGS. 1-7, the x axis represents serum blood glucose readings in subjects orally administered Formulation B or vehicle alone. The Y axis represents the % total patients that the reading represents. The scale is from 0 to 100%. The data for both curves are first ranked from lowest to highest (or highest to lowest). If there are 10 data points, each point would represent 10%. They are then plotted as % vs glucose value.

FIG. 1 shows the survival function without confidence bands of non-diabetic patients on the day before surgery where line #1 refers to subjects treated with Formulation B and line #2 refers to untreated subjects. The p value is 0.62.

FIG. 2 shows the survival function without confidence bands of non-diabetic patients on the third day after surgery where line #1 refers to subjects treated with Formulation B and line #2 refers to untreated subjects. The p value is 0.73.

FIG. 3 shows the survival function without confidence bands of diabetic patients on the day before surgery where line #1 refers to subjects treated with Formulation B and line #2 refers to untreated subjects. The p value is 0.28.

FIG. 4 shows the survival function without confidence bands of diabetic patients on the day of surgery where line #1 refers to subjects treated with Formulation B and line #2 refers to untreated subjects. The p value is 0.89.

FIG. 5 shows the survival function without confidence bands of diabetic patients one day after surgery where line #1 refers to subjects treated with Formulation B and line #2 refers to untreated subjects. The p value is 0.91.

FIG. 6 shows the survival function without confidence bands of diabetic patients two days after surgery where line #1 refers to subjects treated with Formulation B and line #2 refers to untreated subjects. The p value is 0.11.

FIG. 7 shows the survival function without confidence bands of diabetic patients three days after surgery where line #1 refers to subjects treated with Formulation B and line #2 refers to untreated subjects. The p value is 0.0399.

FIG. 8 is a graph showing mean serum glucose levels over the course of 4 days in diabetic subjects treated with Formulation B (solid line) versus vehicle (dashed line), where p is less than 0.05.

FIG. 9 is a graph showing mean serum glucose levels over the course of 4 days in non-diabetic subjects treated with Formulation B versus vehicle.

### DETAILED DESCRIPTION

Unless defined otherwise, technical and scientific terms used herein have the same meaning as commonly understood by a person of ordinary skill in the art. Any methods, devices and materials similar or equivalent to those described herein can be used in the practice of the disclosure. The following definitions are provided to facilitate understanding of certain terms used frequently herein and are not meant to limit the scope of the present disclosure.

“Insulin receptor protease inhibitor” refers to a compound that prevents protease cleavage of an insulin receptor. In embodiments, “insulin receptor protease inhibitor” is a protease inhibitor. In embodiments, the “insulin receptor protease inhibitor” is a serine protease inhibitor, a matrix metalloproteinase inhibitor, or a lipase. In embodiments, the “insulin receptor protease inhibitor” is a serine protease inhibitor. In embodiments, the “insulin receptor protease inhibitor” is tranexamic acid, gabexate mesilate, camostat mesilate, nafamostat mesilate, α-1-anti-trypsin, α-2-macroglobulin, pancreatic lipase, gastric lipase, pancreatic lipase-related protein 1, pancreatic lipase-related protein 2, aprotinin, or a combination of two or more thereof. In embodiments, the “insulin receptor protease inhibitor” is tranexamic acid.

“Patient” or “subject” refer to a living organism suffering from or prone to a disease or condition that can be treated by administration of a compound or pharmaceutical composition or by a method as provided herein. Non-limiting examples include humans, other mammals, bovines, rats, mice, dogs, cats, monkeys, goat, sheep, cows, and other non-mammalian animals. In embodiments, a patient or a subject is a human.

“Diabetic patient” refers to a patient who has Type II diabetes or who has pre-Type II diabetes. Standards for determining whether a patient has Type II diabetes or has pre-Type II diabetes are well known in the art, and described, for example, by the American Diabetes Association®. For example, a diabetic patient may have an A1C of 6.5% or more, a fasting plasma glucose (FPG) of 126 mg/dl or more, or an oral glucose tolerance test (OGTT) measurement of 200 mg/dl or more. For pre-diabetes, a patient may have an A1C of 5.7% to 6.4%, an FPG of 100 mg/dl to 125 mg/dl, or an OGTT of 140 mg/dl to 199 mg/dl. Glucose can be measured by other means including: (i) fasting whole blood glucose test; (ii) fasting plasma glucose test; (iii) fasting serum glucose test; (iv) non-fasting whole blood glucose test; (v) non-fasting plasma glucose test; and (vi) non-fasting serum glucose test.

“Surgical procedure” or “surgery” refers to any surgical procedure known in the art. In embodiments, the “surgical procedure” involves or is near any internal organ, such as the stomach, intestines, colon, liver, kidney, pancreas, and reproductive organs. In embodiments, the “surgical procedure” is a surgical procedure involving the digestive system, excretory system, reproductive system, skeletal system, nervous system, respiratory system, cardiovascular system, skeletal system, and the like. In embodiments, the “surgical procedure” is a gastrointestinal surgical procedure involving the esophagus, stomach, and/or intestines. In embodiments, the “surgical procedure” is cardiovascular surgery, such as coronary artery bypass graft (CABG) surgery, heart valve replacement surgery, cardiopulmonary bypass (CBP), and the like. In embodiments, the “surgical procedure” is a bowel resection, such as a large bowel resection, a small bowel resection, or a colon resection.

“Before a surgical procedure” refers to the time before a surgical procedure. In terms of administering a composition “before a surgical procedure,” the composition is generally administered one day before the day of the surgical procedure. In embodiments, “before a surgical procedure” is not the day the surgical procedure is performed.

“After a surgical procedure” refers to the time after a surgical procedure. In terms of administering a composition “after a surgical procedure,” the composition is generally administered on the same day as, but after, the surgical procedure. In embodiments, “after a surgical procedure” means the composition is administered the day after the surgical procedure (i.e., not on the same day as the surgical procedure). In terms of identifying days “after a surgical procedure,” the day of the surgical procedure may be identified as day 0, and the day immediately after the surgical procedure may be identified as day 1.

“During a surgical procedure” refers to any time after the surgical incision on the skin of the patient and prior to closing of the skin at the surgical site. “During a surgical procedure” includes the time after incision of the skin but before the actual procedure is performed; during the performance of the surgical procedure; after the surgical procedure is completed but before closing or suturing of the skin at the surgical site; or any combination thereof. When the compositions described herein are administered during a surgical procedure, they are generally administered by lavage or by a nasogastric tube or orogastric tube. “Lavage” refers to washing, flushing, or bathing of a surgical site or bodily cavity during a surgical procedure.

“Lowering blood glucose levels” and “improving glycemic control” refer to reducing the level or measurement (when compared to baseline) of one or more of (i) fasting whole blood glucose test; (ii) fasting plasma glucose test; (iii) fasting serum glucose test; (iv) non-fasting whole blood glucose test; (v) non-fasting plasma glucose test; (vi) non-fasting serum glucose test; (vii) OGTT, or (viii) A1C by any amount compared to baseline. In embodiments, the fasting or non-fasting whole blood, plasma, or serum glucose test is reduced by 1 mg/dl, or by 2 mg/dl, or by 3 mg/dl, or by 4 mg/dl, or by 5 mg/dl, or by 6 mg/dl, or by 7 gm/dl, or by 8 mg/dl, or by 9, mg/dl, or by 10 mg/dl, or by 11 mg/dl, or by 12 mg/dl, or by 13 mg/dl, or by 14 mg/dl, or by 15 mg/dl, or by 16 mg/dl, or by 17 mg/dl, or by 18 mg/dl, or by 19 mg/dl, or by 20 mg/dl, or by 21 mg/dl, or by 22 mg/dl, or by 23 mg/dl, or by 24 mg/dl, or by 25 mg/dl, or by 26 mg/dl, or by 27 mg/dl, or by 28 mg/dl, or by 29 mg/dl, or by 30 mg/dl, or by 31 mg/dl, or by 32 mg/dl, or by 33 mg/dl, or by 34 mg/dl, or by 35 mg/dl, or by 36 mg/dl, or by 37 mg/dl, or by 38 mg/dl, or by 39 mg/dl, or by 40 mg/dl, or by 41 mg/dl, or by 42 mg/dl, or by 43 mg/dl, or by 44 mg/dl, or by 45 mg/dl, or by 46 mg/dl, or by 47 mg/dl, or by 48 mg/dl, or by 49 mg/dl, or by 50 mg/dl, or by 51 mg/dl, or by 52 mg/dl, or by 53 mg/dl, or by 54 mg/dl, or by 55 mg/dl, or by 56 mg/dl, or by 57 mg/dl, or by 58 mg/dl, or by 59 mg/dl, or by 60 mg/dl, or by 61 mg/dl, or by 62 mg/dl, or by 63 mg/dl, or by 64 mg/dl, or by 65 mg/dl, or by 66 mg/dl, or by 67 mg/dl, or by 68 mg/dl, or by 69 mg/dl, or by 70 mg/dl, or by 71 mg/dl, or by 72 mg/dl, or by 73 mg/dl, or by 74 mg/dl, or by 75 mg/dl, or by 76 mg/dl, or by 77 mg/dl, or by 78 mg/dl, or by 79 mg/dl, or by 80 mg/dl, or by 81 mg/dl, or by 82 mg/dl, or by 83 mg/dl, or by 84 mg/dl, or by 85 mg/dl, or by 86 mg/dl, or by 87 mg/dl, or by 88 mg/dl, or by 89 mg/dl, or by 90 mg/dl, or by 91 mg/dl, or by 92 mg/dl, or by 93 mg/dl, or by 94 mg/dl, or by 95 mg/dl, or by 96 mg/dl, or by 97 mg/dl, or by 98 mg/dl, or by 99 mg/dl, by 100 mg/dl, or by 101 mg/dl or more. In embodiments, the A1C is reduced to 6.5% or less; or 6.4% or less; or 6.3% or less; or 6.2% or less; or 6.1% or less: or 6% or less.

“Reducing post-surgical blood glucose levels” refers to reducing the level or measurement (when compared to baseline) of one or more of (i) fasting whole blood glucose test; (ii) fasting plasma glucose test; (iii) fasting serum glucose test; (iv) non-fasting whole blood glucose test; (v) non-fasting plasma glucose test; (vi) non-fasting serum glucose test; (vii) OGTT, or (viii) A1C by any amount after the patient undergoes a surgical procedure. In embodiments, blood glucose levels are lowered in about 1 day to about 5 days after the surgical procedure. In embodiments, blood glucose levels are lowered in about 1 day to about 4 days after the surgical procedure. In embodiments, blood glucose levels are lowered in about 1 day to about 3 days after a surgical procedure. In embodiments, blood glucose levels are lowered in about 2 days to about 3 days after a surgical procedure. In embodiments, blood glucose levels are lowered in about 2 days after a surgical procedure. In embodiments, blood glucose levels are lowered in about 3 days after a surgical procedure. In embodiments, the fasting or non-fasting whole blood, plasma, or serum glucose test is reduced by 1 mg/dl, or by 2 mg/dl, or by 3 mg/dl, or by 4 mg/dl, or by 5 mg/dl, or by 6 mg/dl, or by 7 gm/dl, or by 8 mg/dl, or by 9, mg/dl, or by 10 mg/dl, or by 11 mg/dl, or by 12 mg/dl, or by 13 mg/dl, or by 14 mg/dl, or by 15 mg/dl, or by 16 mg/dl, or by 17 mg/dl, or by 18 mg/dl, or by 19 mg/dl, or by 20 mg/dl, or by 21 mg/dl, or by 22 mg/dl, or by 23 mg/dl, or by 24 mg/dl, or by 25 mg/dl, or by 26 mg/dl, or by 27 mg/dl, or by 28 mg/dl, or by 29 mg/dl, or by 30 mg/dl, or by 31 mg/dl, or by 32 mg/dl, or by 33 mg/dl, or by 34 mg/dl, or by 35 mg/dl, or by 36 mg/dl, or by 37 mg/dl, or by 38 mg/dl, or by 39 mg/dl, or by 40 mg/dl, or by 41 mg/dl, or by 42 mg/dl, or by 43 mg/dl, or by 44 mg/dl, or by 45 mg/dl, or by 46 mg/dl, or by 47 mg/dl, or by 48 mg/dl, or by 49 mg/dl, or by 50 mg/dl, or by 51 mg/dl, or by 52 mg/dl, or by 53 mg/dl, or by 54 mg/dl, or by 55 mg/dl, or by 56 mg/dl, or by 57 mg/dl, or by 58 mg/dl, or by 59 mg/dl, or by 60 mg/dl, or by 61 mg/dl, or by 62 mg/dl, or by 63 mg/dl, or by 64 mg/dl, or by 65 mg/dl, or by 66 mg/dl, or by 67 mg/dl, or by 68 mg/dl, or by 69 mg/dl, or by 70 mg/dl, or by 71 mg/dl, or by 72 mg/dl, or by 73 mg/dl, or by 74 mg/dl, or by 75 mg/dl, or by 76 mg/dl, or by 77 mg/dl, or by 78 mg/dl, or by 79 mg/dl, or by 80 mg/dl, or by 81 mg/dl, or by 82 mg/dl, or by 83 mg/dl, or by 84 mg/dl, or by 85 mg/dl, or by 86 mg/dl, or by 87 mg/dl, or by 88 mg/dl, or by 89 mg/dl, or by 90 mg/dl, or by 91 mg/dl, or by 92 mg/dl, or by 93 mg/dl, or by 94 mg/dl, or by 95 mg/dl, or by 96 mg/dl, or by 97 mg/dl, or by 98 mg/dl, or by 99 mg/dl, by 100 mg/dl, or by 101 mg/dl or more. In embodiments, the A1C is reduced to 6.5% or less; or 6.4% or less; or 6.3% or less; or 6.2% or less; or 6.1% or less: or 6% or less.

“Baseline measurement” with reference to blood glucose levels refers to the blood glucose measurement taken prior to administration of the first dose of the pharmaceutical composition described herein. In embodiments, the baseline measurement is the blood glucose measurement taken on the day before surgery and before administration of the first dose of the pharmaceutical composition described herein. In embodiments, the baseline measurement is the blood glucose measurement taken on the day of surgery, and after administration of the first dose of the pharmaceutical composition described herein, but before administration of the second dose of the pharmaceutical composition described herein. In embodiments, the baseline measurement is the blood glucose measurement taken on the day of the surgical procedure and after administration of the second dose of the pharmaceutical composition described herein.

“Administering” generally means oral administration. Alternative modes of administration may be possible, such as lavage, intravenous, parenteral, intraperitoneal, intramuscular, intralesional, intrathecal, intracranial, intranasal or subcutaneous administration. Liquid forms of the compositions described herein include solutions, suspensions, and emulsions, preferably solutions. Solid forms of the compositions described herein include capsules, tablets, and caplets. By “administered chronically” is meant the administration of a composition (e.g., a solid or liquid dosage form) on a periodic basis (e.g., at least once a day, twice a day, three times a day, four times a day, at least once a week, twice a week, three times a week, four times a week, five times a week, six times a week, seven times a week, once a month, twice a month, three times a month, and four times a month) over an extended period of time (e.g., at least one month, two months, three months, four months, five months, six months, seven months, eight months, nine months, ten months, eleven months, one year, two years, three years, four years, five years, six years, seven years, eight years, nine years, ten years, longer, or for lifetime).

“Oral administration” and “orally administering” refers to ingestion of the pharmaceutical compositions described herein by mouth and into the gastrointestinal tract of the patient. Oral administration includes gavage, where the pharmaceutical compositions described herein are administered to a patient through a tube (e.g., nasogastric, orogastric) leading down from the throat and/or nose to the stomach. The term “gavage” is used interchangeably with the term “enteral.” Oral administration also includes ingestion of a solid or liquid dosage form, as described herein.

The terms “treating” or “treatment” refers to any indicia of success in lowering blood glucose levels in diabetic patients.

“Dose” refers to the volume of the pharmaceutical compositions described herein that is administered to the patient, where each of the components (e.g., tranexamic acid) in the pharmaceutical composition are in an amount (e.g., wt %, grams) as described herein. In embodiments, a dose may be administered as a single dose or as a divided dose. Thus, a single dose may be divided into two, three, or four sub-doses and administered over the course of the day. In embodiments, a dose can be administered via a nasogastric or orogastric tube over the course of one to twenty-four hours. For purposes of this disclosure, reference to administering the pharmaceutical composition twice a day refers to dividing a single dose in half (or any other fraction) and administering the divided dose at two different times in a day. Similarly, a dose can be administered via a nasogastric tube, and the same amount of pharmaceutical composition will be administered whether it takes two hours, eight hours, or twenty-four hours to administer the dose to the patient.

A “therapeutically effective amount” is an amount sufficient to accomplish a stated purpose, e.g., reduction of blood glucose levels. A “therapeutically effective amount” of a drug is an amount of a drug that, when administered to a subject, will have the intended effect, e.g., preventing or delaying the onset (or reoccurrence) of an injury, disease, pathology or condition, or reducing the likelihood of the onset (or reoccurrence) of an injury, disease, pathology, or condition, or their symptoms. The full effect does not necessarily occur by administration of one dose and may occur only after administration of a series of doses. Thus, a therapeutically effective amount may be administered in one or more administrations. Determination of a therapeutically effective amount of the compositions described herein is within the capabilities of a skilled artisan, particularly in light of the detailed disclosure herein. As is known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards. Adjusting the dose to achieve maximal efficacy in humans based on the methods described herein and other methods is well within the capabilities of the ordinarily skilled artisan.

The term “pharmaceutically acceptable salts” is meant to include salts of the active compounds, such as electrolytes, that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts.

“About” means a range of values including the specified value, which a person of ordinary skill in the art would consider reasonably similar to the specified value. In embodiments, about means within a standard deviation using measurements generally acceptable in the art. In embodiments, about means a range extending to +/−10%, +/−5%, or +/−1% of the specified value. In embodiments, about means the specified value.

“Molecular weight” or “average molecular weight” refers to number average molecular weight.

In embodiments, the disclosure provides methods for treating diabetes in a patient in need thereof comprising orally administering to the patient a therapeutically effective amount of a pharmaceutical composition; wherein the pharmaceutical composition comprises an insulin receptor protease inhibitor and optionally, (i) polyethylene glycol, (ii) glucose, (iii) electrolytes, (iv) polyethylene glycol and glucose; (v) polyethylene glycol and electrolytes; (vi) glucose and electrolytes; or (vii) polyethylene glycol, glucose, and electrolytes. In embodiments, the disclosure provides methods for improving glycemic control in diabetic patients in a patient in need thereof comprising orally administering to the patient a therapeutically effective amount of a pharmaceutical composition; wherein the pharmaceutical composition comprises an insulin receptor protease inhibitor and optionally, (i) polyethylene glycol, (ii) glucose, (iii) electrolytes, (iv) polyethylene glycol and glucose; (v) polyethylene glycol and electrolytes; (vi) glucose and electrolytes; or (vii) polyethylene glycol, glucose, and electrolytes. In embodiments, the composition comprises an insulin receptor protease inhibitor and polyethylene glycol. In embodiments, the composition comprises an insulin receptor protease inhibitor and glucose. In embodiments, the composition comprises an insulin receptor protease inhibitor and electrolytes. In embodiments, the composition comprises an insulin receptor protease inhibitor, polyethylene glycol, and glucose. In embodiments, the composition comprises an insulin receptor protease inhibitor, polyethylene glycol, glucose, and electrolytes. In embodiments, the composition comprises an insulin receptor protease inhibitor, polyethylene glycol, and electrolytes. In embodiments, the composition comprises an insulin receptor protease inhibitor, glucose, and electrolytes. In embodiments, the composition is orally administered as a liquid dosage form or a solid dosage form. In embodiments, the pharmaceutical compositions are in the form of an aqueous solution.

In embodiments, the disclosure provides methods for lowering blood glucose levels in a diabetic patient in need thereof comprising orally administering to the diabetic patient a therapeutically effective amount of a pharmaceutical composition prior to a surgical procedure to lower blood glucose levels; wherein the pharmaceutical composition comprises an insulin receptor protease inhibitor and optionally, (i) polyethylene glycol, (ii) glucose, (iii) electrolytes, (iv) polyethylene glycol and glucose; (v) polyethylene glycol and electrolytes; (vi) glucose and electrolytes; or (vii) polyethylene glycol, glucose, and electrolytes. In embodiments, the composition comprises an insulin receptor protease inhibitor and polyethylene glycol. In embodiments, the composition comprises an insulin receptor protease inhibitor and glucose. In embodiments, the composition comprises an insulin receptor protease inhibitor and electrolytes. In embodiments, the composition comprises an insulin receptor protease inhibitor, polyethylene glycol, and glucose. In embodiments, the composition comprises an insulin receptor protease inhibitor, polyethylene glycol, glucose, and electrolytes. In embodiments, the composition comprises an insulin receptor protease inhibitor, polyethylene glycol, and electrolytes. In embodiments, the composition comprises an insulin receptor protease inhibitor, glucose, and electrolytes. In embodiments, the methods for lowering blood glucose are methods for reducing post-surgical blood glucose levels. In embodiments, the composition is administered as one or two doses prior to the surgical procedure. In embodiments, the methods further comprise administering the composition to the patient during the surgical procedure. In embodiments, the composition is orally administered as a liquid dosage form or a solid dosage form. In embodiments, the pharmaceutical compositions are in the form of an aqueous solution.

In embodiments, the disclosure provides methods for lowering blood glucose levels in a diabetic patient in need thereof comprising orally administering to the diabetic patient a therapeutically effective amount of a pharmaceutical composition after a surgical procedure to lower blood glucose levels; wherein the pharmaceutical composition comprises an insulin receptor protease inhibitor and optionally, (i) polyethylene glycol, (ii) glucose, (iii) electrolytes, (iv) polyethylene glycol and glucose; (v) polyethylene glycol and electrolytes;

(vi) glucose and electrolytes; or (vii) polyethylene glycol, glucose, and electrolytes. In embodiments, the composition comprises an insulin receptor protease inhibitor and polyethylene glycol. In embodiments, the composition comprises an insulin receptor protease inhibitor and glucose. In embodiments, the composition comprises an insulin receptor protease inhibitor and electrolytes. In embodiments, the composition comprises an insulin receptor protease inhibitor, polyethylene glycol, and glucose. In embodiments, the composition comprises an insulin receptor protease inhibitor, polyethylene glycol, glucose, and electrolytes. In embodiments, the composition comprises an insulin receptor protease inhibitor, polyethylene glycol, and electrolytes. In embodiments, the composition comprises an insulin receptor protease inhibitor, glucose, and electrolytes. In embodiments, the methods for lowering blood glucose are methods for reducing post-surgical blood glucose levels. In embodiments, the composition is administered for one to ten days after the surgical procedure; or from one to nine days after the surgical procedure; or from one to eight days after the surgical procedure; or from one to seven days after the surgical procedure; or from one to six days after the surgical procedure; or from one to five days after the surgical procedure. In embodiments, the composition is administered for one to four days after the surgical procedure. In embodiments, the composition is administered for one to three days after the surgical procedure. In embodiments, the composition is administered for one to two days after the surgical procedure. When administered after the surgical procedure, the compositions may be administered by any means known in the art. For example, the compositions may be administered in one, two, or multiple doses over the course of the day, or may be administered via a nasogastric/orogastric tube over the course of the day (e.g., administered as a drip over a period of time, such as 8 to 24 hours), or administered as a solid dosage form. In embodiments, the methods further comprise administering the composition to the patient during the surgical procedure. In embodiments, the composition is orally administered as a liquid dosage form or a solid dosage form. In embodiments, the pharmaceutical compositions are in the form of an aqueous solution.

In embodiments, the disclosure provides methods for lowering blood glucose levels in a diabetic patient in need thereof comprising orally administering to the diabetic patient a therapeutically effective amount of a pharmaceutical composition prior to a surgical procedure and after a surgical procedure to lower blood glucose levels; wherein the pharmaceutical composition comprises an insulin receptor protease inhibitor and optionally, (i) polyethylene glycol, (ii) glucose, (iii) electrolytes, (iv) polyethylene glycol and glucose; (v) polyethylene glycol and electrolytes; (vi) glucose and electrolytes; or (vii) polyethylene glycol, glucose, and electrolytes. In embodiments, the composition comprises an insulin receptor protease inhibitor and polyethylene glycol. In embodiments, the composition comprises an insulin receptor protease inhibitor and glucose. In embodiments, the composition comprises an insulin receptor protease inhibitor and electrolytes. In embodiments, the composition comprises an insulin receptor protease inhibitor, polyethylene glycol, and glucose. In embodiments, the composition comprises an insulin receptor protease inhibitor, polyethylene glycol, glucose, and electrolytes. In embodiments, the composition comprises an insulin receptor protease inhibitor, polyethylene glycol, and electrolytes. In embodiments, the composition comprises an insulin receptor protease inhibitor, glucose, and electrolytes. In embodiments, the methods for lowering blood glucose are methods for reducing post-surgical blood glucose levels. In embodiments, the composition is administered as one or two doses prior to the surgical procedure. In embodiments, the composition is administered for one to ten days after the surgical procedure; or from one to nine days after the surgical procedure; or from one to eight days after the surgical procedure; or from one to seven days after the surgical procedure; or from one to six days after the surgical procedure; or from one to five days after the surgical procedure. In embodiments, the composition is administered for one to four days after the surgical procedure. In embodiments, the composition is administered for one to three days after the surgical procedure. In embodiments, the composition is administered for one to two days after the surgical procedure. When administered after the surgical procedure, the compositions may be administered by any means known in the art. For example, the compositions may be administered in one, two, or multiple doses over the course of the day, or may be administered via a nasogastric/orogastric tube over the course of the day (e.g., administered as a drip over a period of time, such as 8 to 24 hours). In embodiments, the methods further comprise administering the composition to the patient during the surgical procedure. In embodiments, the composition is orally administered as a liquid dosage form or a solid dosage form. In embodiments, the pharmaceutical compositions are in the form of an aqueous solution.

The methods described provide for lowering blood glucose levels in a diabetic patient by orally administering a therapeutically effective amount of a pharmaceutical composition comprising an insulin receptor protease inhibitor and optionally, (i) polyethylene glycol, (ii) glucose, (iii) electrolytes, (iv) polyethylene glycol and glucose; (v) polyethylene glycol and electrolytes; (vi) glucose and electrolytes; or (vii) polyethylene glycol, glucose, and electrolytes. In embodiments, the disclosure provides methods for treating diabetes in a patient in need thereof comprising orally administering to the patient a therapeutically effective amount of a pharmaceutical composition; wherein the pharmaceutical composition comprises an insulin receptor protease inhibitor and optionally, (i) polyethylene glycol, (ii) glucose, (iii) electrolytes, (iv) polyethylene glycol and glucose; (v) polyethylene glycol and electrolytes; (vi) glucose and electrolytes; or (vii) polyethylene glycol, glucose, and electrolytes. In embodiments, the disclosure provides methods for improving glycemic control in diabetic patients in a patient in need thereof comprising orally administering to the patient a therapeutically effective amount of a pharmaceutical composition; wherein the pharmaceutical composition comprises an insulin receptor protease inhibitor and optionally, (i) polyethylene glycol, (ii) glucose, (iii) electrolytes, (iv) polyethylene glycol and glucose; (v) polyethylene glycol and electrolytes; (vi) glucose and electrolytes; or (vii) polyethylene glycol, glucose, and electrolytes. In embodiments, the composition is administered (a) prior to a surgical procedure, (b) after a surgical procedure, or (c) prior to a surgical procedure and after a surgical procedure. The methods may further comprise administering the composition during the surgical procedure. In embodiments, the composition is administered to a diabetic patient who is not undergoing a surgical procedure or who is in a non-hospital setting. In embodiments, the composition is administered chronically. In these embodiments, the insulin receptor protease inhibitor is a serine protease inhibitor, a matrix metalloproteinase inhibitor, a lipase, or a combination of two or more thereof. In embodiments, the insulin receptor protease inhibitor is a serine protease inhibitor. In embodiments, the insulin receptor protease inhibitor is tranexamic acid, gabexate mesilate, camostat mesilate, nafamostat mesilate, α-1-anti-trypsin, α-2-macroglobulin, pancreatic lipase, gastric lipase, pancreatic lipase-related protein 1, pancreatic lipase-related protein 2, aprotinin, or a combination of two or more thereof. In embodiments, the insulin receptor protease inhibitor is tranexamic acid. In embodiments, the polyethylene glycol has an average molecular weight from about 100 Daltons to about 30,000 Daltons. In embodiments, the polyethylene glycol has an average molecular weight from about 100 Daltons to about 25,000 Daltons. In embodiments, the polyethylene glycol has an average molecular weight from about 500 Daltons to about 20,000 Daltons. In embodiments, the polyethylene glycol has an average molecular weight from about 1,000 Daltons to about 15,000 Daltons. In embodiments, the polyethylene glycol has an average molecular weight from about 1,000 Daltons to about 10,000 Daltons. In embodiments, the polyethylene glycol has an average molecular weight from about 2,000 Daltons to about 5,000 Daltons. In embodiments, the polyethylene glycol has an average molecular weight from about 3,000 Daltons to about 4,000 Daltons. In embodiments, the polyethylene glycol has an average molecular weight of about 3,500 Daltons. In embodiments, the electrolytes comprise sodium, potassium, calcium, magnesium, chloride, bicarbonate, phosphate, sulfate, or a combination of two or more thereof. In embodiments, the electrolytes comprise sodium sulfate, sodium bicarbonate, sodium chloride, potassium chloride, or a combination of two or more thereof. In embodiments the compositions comprise from about 0.1 wt % to about 25 wt % of the insulin receptor protease inhibitor, and optionally, (i) from about 1 wt % to about 25 wt % of the polyethylene glycol, (ii) from about 1 wt % to about 25 wt % of the glucose, (iii) from about 0.001 wt % to about 25 wt % of electrolytes, (iv) the polyethylene glycol and the glucose; (v) the polyethylene glycol and the electrolytes; (vi) the glucose and the electrolytes; or (vii) the polyethylene glycol, the glucose, and the electrolytes. In embodiments the compositions comprise from about 0.1 wt % to about 10 wt % of the insulin receptor protease inhibitor, and, optionally, (i) from about 1 wt % to about 15 wt % of the polyethylene glycol, (ii) from about 1 wt % to about 15 wt % of the glucose, (iii) from about 0.05 wt % to about 20 wt % of electrolytes, (iv) the polyethylene glycol and the glucose; (v) the polyethylene glycol and the electrolytes; (vi) the glucose and the electrolytes; or (vii) the polyethylene glycol, the glucose, and the electrolytes. In embodiments the compositions comprise from about 0.1 wt % to about 5 wt % of the insulin receptor protease inhibitor, and optionally, (i) from about 1 wt % to about 10 wt % of the polyethylene glycol, (ii) from about 1 wt % to about 10 wt % of the glucose, (iii) from about 0.05 wt % to about 5 wt % of electrolytes, (iv) the polyethylene glycol and the glucose; (v) the polyethylene glycol and the electrolytes; (vi) the glucose and the electrolytes; or (vii) the polyethylene glycol, the glucose, and the electrolytes. In embodiments the compositions comprise from about 0.5 wt % to about 2 wt % of the insulin receptor protease inhibitor, and optionally, (i) from about 2 wt % to about 6 wt % of the polyethylene glycol, (ii) from about 2 wt % to about 6 wt % of the glucose, (iii) from about 0.1 wt % to about 2 wt % of electrolytes, (iv) the polyethylene glycol and the glucose; (v) the polyethylene glycol and the electrolytes; (vi) the glucose and the electrolytes; or (vii) the polyethylene glycol, the glucose, and the electrolytes. In embodiments the compositions comprise from about 0.3 wt % to about 2.0 wt % of the insulin receptor protease inhibitor, and optionally, (i) from about 3.5 wt % to about 5.3 wt % of the polyethylene glycol, (ii) from about 3.3 wt % to about 5.7 wt % of the glucose, (iii) from about 0.09 wt % to about 1.6 wt % of electrolytes, (iv) the polyethylene glycol and the glucose; (v) the polyethylene glycol and the electrolytes; (vi) the glucose and the electrolytes; or (vii) the polyethylene glycol, the glucose, and the electrolytes. In embodiments the compositions comprise from about 0.4 wt % to about 1.9 wt % of the insulin receptor protease inhibitor, and optionally, (i) from about 3.6 wt % to about 5.2 wt % of the polyethylene glycol, (ii) from about 3.4 wt % to about 5.6 wt % of the glucose, (iii) from about 0.1 wt % to about 1.5 wt % of electrolytes, (iv) the polyethylene glycol and the glucose; (v) the polyethylene glycol and the electrolytes; (vi) the glucose and the electrolytes; or (vii) the polyethylene glycol, the glucose, and the electrolytes. In embodiments the compositions comprise from about 0.5 wt % to about 1.8 wt % of the insulin receptor protease inhibitor, and optionally, (i) from about 3.7 wt % to about 5.1 wt % of the polyethylene glycol, (ii) from about 3.5 wt % to about 5.5 wt % of the glucose, (iii) from about 0.2 wt % to about 1.4 wt % of electrolytes, (iv) the polyethylene glycol and the glucose; (v) the polyethylene glycol and the electrolytes; (vi) the glucose and the electrolytes; or (vii) the polyethylene glycol, the glucose, and the electrolytes. In embodiments the compositions comprise from about 0.6 wt % to about 1.6 wt % of the insulin receptor protease inhibitor, and optionally (i) from about 3.8 wt % to about 5.0 wt % of the polyethylene glycol, (ii) from about 3.6 wt % to about 5.4 wt % of the glucose, (iii) from about 0.3 wt % to about 1.3 wt % of electrolytes, (iv) the polyethylene glycol and the glucose; (v) the polyethylene glycol and the electrolytes; (vi) the glucose and the electrolytes; or (vii) the polyethylene glycol, the glucose, and the electrolytes. In embodiments the compositions comprise from about 0.7 wt % to about 1.5 wt % of the insulin receptor protease inhibitor, and optionally, (i) from about 3.9 wt % to about 4.9 wt % of the polyethylene glycol, (ii) from about 3.7 wt % to about 5.3 wt % of the glucose, (iii) from about 0.4 wt % to about 1.2 wt % of electrolytes, (iv) the polyethylene glycol and the glucose; (v) the polyethylene glycol and the electrolytes; (vi) the glucose and the electrolytes; or (vii) the polyethylene glycol, the glucose, and the electrolytes. In embodiments the compositions comprise from about 0.8 wt % to about 1.4 wt % of the insulin receptor protease inhibitor, and optionally, (i) from about 4.0 wt % to about 4.8 wt % of the polyethylene glycol, (ii) from about 3.8 wt % to about 5.2 wt % of the glucose, (iii) from about 0.5 wt % to about 1.1 wt % of electrolytes, (iv) the polyethylene glycol and the glucose; (v) the polyethylene glycol and the electrolytes; (vi) the glucose and the electrolytes; or (vii) the polyethylene glycol, the glucose, and the electrolytes. In embodiments the compositions comprise from about 0.9 wt % to about 1.3 wt % of the insulin receptor protease inhibitor, and optionally, (i) from about 4.1 wt % to about 4.7 wt % of the polyethylene glycol, (ii) from about 3.9 wt % to about 5.1 wt % of the glucose, (iii) from about 0.6 wt % to about 1.0 wt % of electrolytes, (iv) the polyethylene glycol and the glucose; (v) the polyethylene glycol and the electrolytes; (vi) the glucose and the electrolytes; or (vii) the polyethylene glycol, the glucose, and the electrolytes. In embodiments the compositions comprise from about 1.0 wt % to about 1.2 wt % of the insulin receptor protease inhibitor, and optionally, (i) from about 4.2 wt % to about 4.6 wt % of the polyethylene glycol, (ii) from about 4.0 wt % to about 5.0 wt % of the glucose, (iii) from about 0.67 wt % to about 0.96 wt % of electrolytes, (iv) the polyethylene glycol and the glucose; (v) the polyethylene glycol and the electrolytes; (vi) the glucose and the electrolytes; or (vii) the polyethylene glycol, the glucose, and the electrolytes. In embodiments the compositions comprise about 1.1 wt % of the insulin receptor protease inhibitor, and optionally, (i) about 4.6 wt % of the polyethylene glycol, (ii) about 4.0 wt % of the glucose, (iii) about 0.96 wt % of electrolytes, (iv) the polyethylene glycol and the glucose; (v) the polyethylene glycol and the electrolytes; (vi) the glucose and the electrolytes; or (vii) the polyethylene glycol, the glucose, and the electrolytes. In embodiments the compositions comprise about 1.1 wt % of the insulin receptor protease inhibitor, and optionally, (i) about 4.2 wt % of the polyethylene glycol, (ii) about 5.0 wt % of the glucose, (iii) about 0.67 wt % of electrolytes, (iv) the polyethylene glycol and the glucose; (v) the polyethylene glycol and the electrolytes; (vi) the glucose and the electrolytes; or (vii) the polyethylene glycol, the glucose, and the electrolytes. As described herein, the pharmaceutical compositions may comprise: (a) an insulin receptor protease inhibitor and polyethylene glycol; (b) an insulin receptor protease inhibitor and glucose; (c) an insulin receptor protease inhibitor and electrolytes; (d) an insulin receptor protease inhibitor, polyethylene glycol, and glucose; (e) an insulin receptor protease inhibitor, polyethylene glycol, glucose, and electrolytes, (0 an insulin receptor protease inhibitor, polyethylene glycol, and electrolytes; or (g) an insulin receptor protease inhibitor, glucose, and electrolytes. In embodiments the compositions may comprise other pharmaceutically acceptable excipients known in the art. In embodiments, the composition is orally administered as a liquid dosage form or a solid dosage form.

In embodiments, the disclosure provides methods for lowering blood glucose levels in a diabetic patient in need thereof comprising orally administering to the diabetic patient a therapeutically effective amount of a pharmaceutical composition prior to a surgical procedure to lower blood glucose levels; wherein the pharmaceutical composition comprises from about 0.01 wt % to about 10 wt % of tranexamic acid, from about 1 wt % to about 10 wt % of polyethylene glycol, from about 1 wt % to about 10 wt % of glucose, and from about 0 wt % to about 10 wt % of electrolytes. In embodiments, the methods for lowering blood glucose are methods for reducing post-surgical blood glucose levels. In embodiments, the composition is administered as one or two doses prior to the surgical procedure. In embodiments, the methods further comprise administering the composition to the patient during the surgical procedure. In embodiments, the composition is orally administered as a liquid dosage form or a solid dosage form. In embodiments, the pharmaceutical compositions are in the form of an aqueous solution. In embodiments, the pharmaceutical composition comprises from about 0.1 wt % to about 8 wt % of tranexamic acid, from about 2 wt % to about 8 wt % of polyethylene glycol, from about 2 wt % to about 8 wt % of glucose, and from about 0.01 wt % to about 8 wt % of electrolytes. In embodiments, the pharmaceutical composition comprises from about 0.1 wt % to about 3 wt % of tranexamic acid, from about 3 wt % to about 6 wt % of the polyethylene glycol, from about 3 wt % to about 5 wt % of the glucose, and from about 0.1 wt % to about 3 wt % of electrolytes. In embodiments, the pharmaceutical composition comprises from about 0.3 wt % to about 2.0 wt % of tranexamic acid, from about 3.8 wt % to about 5.4 wt % of the polyethylene glycol, from about 3.2 wt % to about 4.8 wt % of the glucose, and from about 0.2 wt % to about 1.7 wt % of electrolytes. In embodiments the compositions comprise from about 0.4 wt % to about 1.9 wt % of tranexamic acid, from about 3.9 wt % to about 5.3 wt % of the polyethylene glycol, from about 3.3 wt % to about 4.7 wt % of the glucose, and from about 0.3 wt % to about 1.6 wt % of electrolytes. In embodiments the compositions comprise from about 0.5 wt % to about 1.8 wt % of tranexamic acid, from about 4.0 wt % to about 5.2 wt % of the polyethylene glycol, from about 3.4 wt % to about 4.6 wt % of the glucose, and from about 0.4 wt % to about 1.5 wt % of electrolytes. In embodiments the compositions comprise from about 0.6 wt % to about 1.6 wt % of tranexamic acid, from about 4.1 wt % to about 5.1 wt % of the polyethylene glycol, from about 3.5 wt % to about 4.5 wt % of the glucose, and from about 0.5 wt % to about 1.4 wt % of electrolytes. In embodiments the compositions comprise from about 0.7 wt % to about 1.5 wt % of tranexamic acid, from about 4.2 wt % to about 5.0 wt % of the polyethylene glycol, from about 3.6 wt % to about 4.4 wt % of the glucose, and from about 0.6 wt % to about 1.3 wt % of electrolytes. In embodiments the compositions comprise from about 0.8 wt % to about 1.4 wt % of tranexamic acid, from about 4.3 wt % to about 4.9 wt % of the polyethylene glycol, from about 3.7 wt % to about 4.3 wt % of the glucose, and from about 0.7 wt % to about 1.2 wt % of electrolytes. In embodiments the compositions comprise from about 0.9 wt % to about 1.3 wt % of tranexamic acid, and from about 4.4 wt % to about 4.8 wt % of the polyethylene glycol, from about 3.8 wt % to about 4.2 wt % of the glucose, and from about 0.8 wt % to about 1.1 wt % of electrolytes. In embodiments the compositions comprise from about 1.0 wt % to about 1.2 wt % of tranexamic acid, from about 4.5 wt % to about 4.7 wt % of the polyethylene glycol, from about 3.9 wt % to about 4.1 wt % of the glucose, and from about 0.9 wt % to about 1.0 wt % of electrolytes. In embodiments the compositions comprise about 1.1 wt % of tranexamic acid, about 4.6 wt % of the polyethylene glycol, about 4.0 wt % of the glucose, and about 0.96 wt % of electrolytes. In embodiments, the polyethylene glycol has an average molecular weight from about 1,000 Daltons to about 10,000 Daltons. In embodiments, the polyethylene glycol has an average molecular weight from about 2,000 Daltons to about 5,000 Daltons. In embodiments, the polyethylene glycol has an average molecular weight from about 3,000 Daltons to about 4,000 Daltons. In embodiments, the polyethylene glycol has an average molecular weight of about 3,500 Daltons. In embodiments, the electrolytes comprise sodium, potassium, calcium, magnesium, chloride, bicarbonate, phosphate, sulfate, or a combination of two or more thereof. In embodiments, the electrolytes comprise sodium sulfate, sodium bicarbonate, sodium chloride, potassium chloride, or a combination of two or more thereof. In embodiments, the electrolytes comprise sodium sulfate, sodium bicarbonate, sodium chloride, and potassium chloride.

In embodiments, the disclosure provides methods for lowering blood glucose levels in a diabetic patient in need thereof comprising orally administering to the diabetic patient a therapeutically effective amount of a pharmaceutical composition after a surgical procedure to lower blood glucose levels; wherein the pharmaceutical composition comprises from about 0.01 wt % to about 10 wt % of tranexamic acid, from about 1 wt % to about 10 wt % of polyethylene glycol, from about 1 wt % to about 10 wt % of glucose, and from about 0 wt % to about 10 wt % of electrolytes. In embodiments, the methods for lowering blood glucose are methods for reducing post-surgical blood glucose levels. In embodiments, the composition is administered for one to ten days after the surgical procedure; or from one to nine days after the surgical procedure; or from one to eight days after the surgical procedure; or from one to seven days after the surgical procedure; or from one to six days after the surgical procedure; or from one to five days after the surgical procedure. In embodiments, the composition is administered for one to four days after the surgical procedure. In embodiments, the composition is administered for one to three days after the surgical procedure. In embodiments, the composition is administered for one to two days after the surgical procedure. When administered after the surgical procedure, the compositions may be administered by any means known in the art. For example, the compositions may be administered in one, two, or multiple doses over the course of the day, or may be administered via a nasogastric/orogastric tube over the course of the day (e.g., administered as a drip over a period of time, such as 8 to 24 hours). In embodiments, the methods further comprise administering the composition to the patient during the surgical procedure. In embodiments, the composition is orally administered as a liquid dosage form or a solid dosage form. In embodiments, the pharmaceutical compositions are in the form of an aqueous solution. In embodiments, the pharmaceutical composition comprises from about 0.1 wt % to about 8 wt % of tranexamic acid, from about 2 wt % to about 8 wt % of polyethylene glycol, from about 2 wt % to about 8 wt % of glucose, and from about 0.01 wt % to about 8 wt % of electrolytes. In embodiments, the pharmaceutical composition comprises from about 0.1 wt % to about 3 wt % of tranexamic acid, from about 3 wt % to about 6 wt % of the polyethylene glycol, from about 3 wt % to about 5 wt % of the glucose, and from about 0.1 wt % to about 3 wt % of electrolytes. In embodiments, the pharmaceutical composition comprises from about 0.3 wt % to about 2.0 wt % of tranexamic acid, from about 3.8 wt % to about 5.4 wt % of the polyethylene glycol, from about 3.2 wt % to about 4.8 wt % of the glucose, and from about 0.2 wt % to about 1.7 wt % of electrolytes. In embodiments the compositions comprise from about 0.4 wt % to about 1.9 wt % of tranexamic acid, from about 3.9 wt % to about 5.3 wt % of the polyethylene glycol, from about 3.3 wt % to about 4.7 wt % of the glucose, and from about 0.3 wt % to about 1.6 wt % of electrolytes. In embodiments the compositions comprise from about 0.5 wt % to about 1.8 wt % of tranexamic acid, from about 4.0 wt % to about 5.2 wt % of the polyethylene glycol, from about 3.4 wt % to about 4.6 wt % of the glucose, and from about 0.4 wt % to about 1.5 wt % of electrolytes. In embodiments the compositions comprise from about 0.6 wt % to about 1.6 wt % of tranexamic acid, from about 4.1 wt % to about 5.1 wt % of the polyethylene glycol, from about 3.5 wt % to about 4.5 wt % of the glucose, and from about 0.5 wt % to about 1.4 wt % of electrolytes. In embodiments the compositions comprise from about 0.7 wt % to about 1.5 wt % of tranexamic acid, from about 4.2 wt % to about 5.0 wt % of the polyethylene glycol, from about 3.6 wt % to about 4.4 wt % of the glucose, and from about 0.6 wt % to about 1.3 wt % of electrolytes. In embodiments the compositions comprise from about 0.8 wt % to about 1.4 wt % of tranexamic acid, from about 4.3 wt % to about 4.9 wt % of the polyethylene glycol, from about 3.7 wt % to about 4.3 wt % of the glucose, and from about 0.7 wt % to about 1.2 wt % of electrolytes. In embodiments the compositions comprise from about 0.9 wt % to about 1.3 wt % of tranexamic acid, and from about 4.4 wt % to about 4.8 wt % of the polyethylene glycol, from about 3.8 wt % to about 4.2 wt % of the glucose, and from about 0.8 wt % to about 1.1 wt % of electrolytes. In embodiments the compositions comprise from about 1.0 wt % to about 1.2 wt % of tranexamic acid, from about 4.5 wt % to about 4.7 wt % of the polyethylene glycol, from about 3.9 wt % to about 4.1 wt % of the glucose, and from about 0.9 wt % to about 1.0 wt % of electrolytes. In embodiments the compositions comprise about 1.1 wt % of tranexamic acid, about 4.6 wt % of the polyethylene glycol, about 4.0 wt % of the glucose, and about 0.96 wt % of electrolytes. In embodiments, the polyethylene glycol has an average molecular weight from about 1,000 Daltons to about 10,000 Daltons. In embodiments, the polyethylene glycol has an average molecular weight from about 2,000 Daltons to about 5,000 Daltons. In embodiments, the polyethylene glycol has an average molecular weight from about 3,000 Daltons to about 4,000 Daltons. In embodiments, the polyethylene glycol has an average molecular weight of about 3,500 Daltons. In embodiments, the electrolytes comprise sodium, potassium, calcium, magnesium, chloride, bicarbonate, phosphate, sulfate, or a combination of two or more thereof. In embodiments, the electrolytes comprise sodium sulfate, sodium bicarbonate, sodium chloride, potassium chloride, or a combination of two or more thereof. In embodiments, the electrolytes comprise sodium sulfate, sodium bicarbonate, sodium chloride, and potassium chloride.

In embodiments, the disclosure provides methods for lowering blood glucose levels in a diabetic patient in need thereof comprising orally administering to the diabetic patient a therapeutically effective amount of a pharmaceutical composition prior to a surgical procedure and after a surgical procedure to lower blood glucose levels; wherein the pharmaceutical composition comprises from about 0.01 wt % to about 10 wt % of tranexamic acid, from about 1 wt % to about 10 wt % of polyethylene glycol, from about 1 wt % to about 10 wt % of glucose, and from about 0 wt % to about 10 wt % of electrolytes. In embodiments, the methods for lowering blood glucose are methods for reducing post-surgical blood glucose levels. In embodiments, the composition is administered as one or two doses prior to the surgical procedure. In embodiments, the composition is administered for one to ten days after the surgical procedure; or from one to nine days after the surgical procedure; or from one to eight days after the surgical procedure; or from one to seven days after the surgical procedure; or from one to six days after the surgical procedure; or from one to five days after the surgical procedure. In embodiments, the composition is administered for one to four days after the surgical procedure. In embodiments, the composition is administered for one to three days after the surgical procedure. In embodiments, the composition is administered for one to two days after the surgical procedure. When administered after the surgical procedure, the compositions may be administered by any means known in the art. For example, the compositions may be administered in one, two, or multiple doses over the course of the day, or may be administered via a nasogastric/orogastric tube over the course of the day (e.g., administered as a drip over a period of time, such as 8 to 24 hours). In embodiments, the methods further comprise administering the composition to the patient during the surgical procedure. In embodiments, the composition is orally administered as a liquid dosage form or a solid dosage form. In embodiments, the pharmaceutical compositions are in the form of an aqueous solution. In embodiments, the pharmaceutical composition comprises from about 0.1 wt % to about 8 wt % of tranexamic acid, from about 2 wt % to about 8 wt % of polyethylene glycol, from about 2 wt % to about 8 wt % of glucose, and from about 0.01 wt % to about 8 wt % of electrolytes. In embodiments, the pharmaceutical composition comprises from about 0.1 wt % to about 3 wt % of tranexamic acid, from about 3 wt % to about 6 wt % of the polyethylene glycol, from about 3 wt % to about 5 wt % of the glucose, and from about 0.1 wt % to about 3 wt % of electrolytes. In embodiments, the pharmaceutical composition comprises from about 0.3 wt % to about 2.0 wt % of tranexamic acid, from about 3.8 wt % to about 5.4 wt % of the polyethylene glycol, from about 3.2 wt % to about 4.8 wt % of the glucose, and from about 0.2 wt % to about 1.7 wt % of electrolytes. In embodiments the compositions comprise from about 0.4 wt % to about 1.9 wt % of tranexamic acid, from about 3.9 wt % to about 5.3 wt % of the polyethylene glycol, from about 3.3 wt % to about 4.7 wt % of the glucose, and from about 0.3 wt % to about 1.6 wt % of electrolytes. In embodiments the compositions comprise from about 0.5 wt % to about 1.8 wt % of tranexamic acid, from about 4.0 wt % to about 5.2 wt % of the polyethylene glycol, from about 3.4 wt % to about 4.6 wt % of the glucose, and from about 0.4 wt % to about 1.5 wt % of electrolytes. In embodiments the compositions comprise from about 0.6 wt % to about 1.6 wt % of tranexamic acid, from about 4.1 wt % to about 5.1 wt % of the polyethylene glycol, from about 3.5 wt % to about 4.5 wt % of the glucose, and from about 0.5 wt % to about 1.4 wt % of electrolytes. In embodiments the compositions comprise from about 0.7 wt % to about 1.5 wt % of tranexamic acid, from about 4.2 wt % to about 5.0 wt % of the polyethylene glycol, from about 3.6 wt % to about 4.4 wt % of the glucose, and from about 0.6 wt % to about 1.3 wt % of electrolytes. In embodiments the compositions comprise from about 0.8 wt % to about 1.4 wt % of tranexamic acid, from about 4.3 wt % to about 4.9 wt % of the polyethylene glycol, from about 3.7 wt % to about 4.3 wt % of the glucose, and from about 0.7 wt % to about 1.2 wt % of electrolytes. In embodiments the compositions comprise from about 0.9 wt % to about 1.3 wt % of tranexamic acid, and from about 4.4 wt % to about 4.8 wt % of the polyethylene glycol, from about 3.8 wt % to about 4.2 wt % of the glucose, and from about 0.8 wt % to about 1.1 wt % of electrolytes. In embodiments the compositions comprise from about 1.0 wt % to about 1.2 wt % of tranexamic acid, from about 4.5 wt % to about 4.7 wt % of the polyethylene glycol, from about 3.9 wt % to about 4.1 wt % of the glucose, and from about 0.9 wt % to about 1.0 wt % of electrolytes. In embodiments the compositions comprise about 1.1 wt % of tranexamic acid, about 4.6 wt % of the polyethylene glycol, about 4.0 wt % of the glucose, and about 0.96 wt % of electrolytes. In embodiments, the polyethylene glycol has an average molecular weight from about 1,000 Daltons to about 10,000 Daltons. In embodiments, the polyethylene glycol has an average molecular weight from about 2,000 Daltons to about 5,000 Daltons. In embodiments, the polyethylene glycol has an average molecular weight from about 3,000 Daltons to about 4,000 Daltons. In embodiments, the polyethylene glycol has an average molecular weight of about 3,500 Daltons. In embodiments, the electrolytes comprise sodium, potassium, calcium, magnesium, chloride, bicarbonate, phosphate, sulfate, or a combination of two or more thereof. In embodiments, the electrolytes comprise sodium sulfate, sodium bicarbonate, sodium chloride, potassium chloride, or a combination of two or more thereof. In embodiments, the electrolytes comprise sodium sulfate, sodium bicarbonate, sodium chloride, and potassium chloride.

In embodiments, the disclosure provides methods for lowering blood glucose levels in a diabetic patient in need thereof comprising orally administering to the diabetic patient a therapeutically effective amount of a pharmaceutical composition prior to a surgical procedure to lower blood glucose levels; wherein the aqueous pharmaceutical composition comprises from about 0.01 wt % to about 10 wt % of tranexamic acid, from about 1 wt % to about 10 wt % of polyethylene glycol, from about 1 wt % to about 10 wt % of glucose, and from about 0 wt % to about 10 wt % of electrolytes. In embodiments, the methods for lowering blood glucose are methods for reducing post-surgical blood glucose levels. In embodiments, the composition is administered as one or two doses prior to the surgical procedure. In embodiments, the methods further comprise administering the composition to the patient during the surgical procedure. In embodiments, the composition is orally administered as a liquid dosage form or a solid dosage form. In embodiments, the pharmaceutical compositions are in the form of an aqueous solution. In embodiments, the pharmaceutical comprises from about 0.1 wt % to about 5 wt % of tranexamic acid, from about 2 wt % to about 8 wt % of polyethylene glycol, from about 2 wt % to about 8 wt % of glucose, and from about 0.001 wt % to about 8 wt % of electrolytes. In embodiments, the pharmaceutical composition comprises from about 0.1 wt % to about 3 wt % of tranexamic acid, from about 2 wt % to about 6 wt % of polyethylene glycol, from about 3 wt % to about 7 wt % of glucose, and from about 0.01 wt % to about 3 wt % of electrolytes. In embodiments, the pharmaceutical composition comprises from about 0.3 wt % to about 2.0 wt % of tranexamic acid, from about 3.4 wt % to about 5.0 wt % of polyethylene glycol, from about 4.2 wt % to about 5.8 wt % of glucose, and from about 0.08 wt % to about 1.2 wt % of electrolytes. In embodiments the compositions comprise from about 0.4 wt % to about 1.9 wt % of tranexamic acid, from about 3.5 wt % to about 4.9 wt % of the polyethylene glycol, from about 4.3 wt % to about 5.7 wt % of the glucose, and from about 0.09 wt % to about 1.3 wt % of electrolytes. In embodiments the compositions comprise from about 0.5 wt % to about 1.8 wt % of tranexamic acid, from about 3.6 wt % to about 4.8 wt % of the polyethylene glycol, from about 4.4 wt % to about 5.6 wt % of the glucose, and from about 0.1 wt % to about 1.2 wt % of electrolytes. In embodiments the compositions comprise from about 0.6 wt % to about 1.6 wt % of tranexamic acid, from about 3.7 wt % to about 4.7 wt % of the polyethylene glycol, from about 4.5 wt % to about 5.5 wt % of the glucose, and from about 0.2 wt % to about 1.1 wt % of electrolytes. In embodiments the compositions comprise from about 0.7 wt % to about 1.5 wt % of tranexamic acid, from about 3.8 wt % to about 4.6 wt % of the polyethylene glycol, from about 4.6 wt % to about 5.4 wt % of the glucose, and from about 0.3 wt % to about 1.0 wt % of electrolytes. In embodiments the compositions comprise from about 0.8 wt % to about 1.4 wt % of tranexamic acid, from about 3.9 wt % to about 4.5 wt % of the polyethylene glycol, from about 4.7 wt % to about 5.3 wt % of the glucose, and from about 0.4 wt % to about 0.9 wt % of electrolytes. In embodiments the compositions comprise from about 0.9 wt % to about 1.3 wt % of tranexamic acid, and from about 4.0 wt % to about 4.4 wt % of the polyethylene glycol, from about 4.8 wt % to about 5.2 wt % of the glucose, and from about 0.5 wt % to about 0.8 wt % of electrolytes. In embodiments the compositions comprise from about 1.0 wt % to about 1.2 wt % of tranexamic acid, from about 4.1 wt % to about 4.3 wt % of the polyethylene glycol, from about 4.9 wt % to about 5.1 wt % of the glucose, and from about 0.6 wt % to about 0.7 wt % of electrolytes. In embodiments the compositions comprise about 1.1 wt % of tranexamic acid, about 4.2 wt % of the polyethylene glycol, about 5.0 wt % of the glucose, and about 0.67 wt % of electrolytes. In embodiments, the polyethylene glycol has an average molecular weight from about 1,000 Daltons to about 10,000 Daltons. In embodiments, the polyethylene glycol has an average molecular weight from about 2,000 Daltons to about 5,000 Daltons. In embodiments, the polyethylene glycol has an average molecular weight from about 3,000 Daltons to about 4,000 Daltons. In embodiments, the polyethylene glycol has an average molecular weight of about 3,500 Daltons. In embodiments, the electrolytes comprise sodium, potassium, calcium, magnesium, chloride, bicarbonate, phosphate, sulfate, or a combination of two or more thereof. In embodiments, the electrolytes comprise sodium sulfate, sodium bicarbonate, sodium chloride, potassium chloride, or a combination of two or more thereof. In embodiments, the electrolytes comprise sodium sulfate, sodium bicarbonate, sodium chloride, and potassium chloride.

In embodiments, the disclosure provides methods for lowering blood glucose levels in a diabetic patient in need thereof comprising orally administering to the diabetic patient a therapeutically effective amount of a pharmaceutical composition after a surgical procedure to lower blood glucose levels; wherein the pharmaceutical composition comprises from about 0.01 wt % to about 10 wt % of tranexamic acid, from about 1 wt % to about 10 wt % of polyethylene glycol, from about 1 wt % to about 10 wt % of glucose, and from about 0 wt % to about 10 wt % of electrolytes. In embodiments, the methods for lowering blood glucose are methods for reducing post-surgical blood glucose levels. In embodiments, the composition is administered for one to ten days after the surgical procedure; or from one to nine days after the surgical procedure; or from one to eight days after the surgical procedure; or from one to seven days after the surgical procedure; or from one to six days after the surgical procedure;

or from one to five days after the surgical procedure. In embodiments, the composition is administered for one to four days after the surgical procedure. In embodiments, the composition is administered for one to three days after the surgical procedure. In embodiments, the composition is administered for one to two days after the surgical procedure. When administered after the surgical procedure, the compositions may be administered by any means known in the art. For example, the compositions may be administered in one, two, or multiple doses over the course of the day, or may be administered via a nasogastric/orogastric tube over the course of the day (e.g., administered as a drip over a period of time, such as 8 to 24 hours). In embodiments, the methods further comprise administering the composition to the patient during the surgical procedure. In embodiments, the composition is orally administered as a liquid dosage form or a solid dosage form. In embodiments, the pharmaceutical compositions are in the form of an aqueous solution. In embodiments, the pharmaceutical comprises from about 0.1 wt % to about 5 wt % of tranexamic acid, from about 2 wt % to about 8 wt % of polyethylene glycol, from about 2 wt % to about 8 wt % of glucose, and from about 0.001 wt % to about 8 wt % of electrolytes. In embodiments, the pharmaceutical comprises from about 0.1 wt % to about 3 wt % of tranexamic acid, from about 2 wt % to about 6 wt % of polyethylene glycol, from about 3 wt % to about 7 wt % of glucose, and from about 0.01 wt % to about 3 wt % of electrolytes. In embodiments, the pharmaceutical comprises from about 0.3 wt % to about 2.0 wt % of tranexamic acid, from about 3.4 wt % to about 5.0 wt % of polyethylene glycol, from about 4.2 wt % to about 5.8 wt % of glucose, and from about 0.08 wt % to about 1.2 wt % of electrolytes. In embodiments the compositions comprise from about 0.4 wt % to about 1.9 wt % of tranexamic acid, from about 3.5 wt % to about 4.9 wt % of the polyethylene glycol, from about 4.3 wt % to about 5.7 wt % of the glucose, and from about 0.09 wt % to about 1.3 wt % of electrolytes. In embodiments the compositions comprise from about 0.5 wt % to about 1.8 wt % of tranexamic acid, from about 3.6 wt % to about 4.8 wt % of the polyethylene glycol, from about 4.4 wt % to about 5.6 wt % of the glucose, and from about 0.1 wt % to about 1.2 wt % of electrolytes. In embodiments the compositions comprise from about 0.6 wt % to about 1.6 wt % of tranexamic acid, from about 3.7 wt % to about 4.7 wt % of the polyethylene glycol, from about 4.5 wt % to about 5.5 wt % of the glucose, and from about 0.2 wt % to about 1.1 wt % of electrolytes. In embodiments the compositions comprise from about 0.7 wt % to about 1.5 wt % of tranexamic acid, from about 3.8 wt % to about 4.6 wt % of the polyethylene glycol, from about 4.6 wt % to about 5.4 wt % of the glucose, and from about 0.3 wt % to about 1.0 wt % of electrolytes. In embodiments the compositions comprise from about 0.8 wt % to about 1.4 wt % of tranexamic acid, from about 3.9 wt % to about 4.5 wt % of the polyethylene glycol, from about 4.7 wt % to about 5.3 wt % of the glucose, and from about 0.4 wt % to about 0.9 wt % of electrolytes. In embodiments the compositions comprise from about 0.9 wt % to about 1.3 wt % of tranexamic acid, and from about 4.0 wt % to about 4.4 wt % of the polyethylene glycol, from about 4.8 wt % to about 5.2 wt % of the glucose, and from about 0.5 wt % to about 0.8 wt % of electrolytes. In embodiments the compositions comprise from about 1.0 wt % to about 1.2 wt % of tranexamic acid, from about 4.1 wt % to about 4.3 wt % of the polyethylene glycol, from about 4.9 wt % to about 5.1 wt % of the glucose, and from about 0.6 wt % to about 0.7 wt % of electrolytes. In embodiments the compositions comprise about 1.1 wt % of tranexamic acid, about 4.2 wt % of the polyethylene glycol, about 5.0 wt % of the glucose, and about 0.67 wt % of electrolytes. In embodiments, the polyethylene glycol has an average molecular weight from about 1,000 Daltons to about 10,000 Daltons. In embodiments, the polyethylene glycol has an average molecular weight from about 2,000 Daltons to about 5,000 Daltons. In embodiments, the polyethylene glycol has an average molecular weight from about 3,000 Daltons to about 4,000 Daltons. In embodiments, the polyethylene glycol has an average molecular weight of about 3,500 Daltons. In embodiments, the electrolytes comprise sodium, potassium, calcium, magnesium, chloride, bicarbonate, phosphate, sulfate, or a combination of two or more thereof. In embodiments, the electrolytes comprise sodium sulfate, sodium bicarbonate, sodium chloride, potassium chloride, or a combination of two or more thereof. In embodiments, the electrolytes comprise sodium sulfate, sodium bicarbonate, sodium chloride, and potassium chloride.

In embodiments, the disclosure provides methods for lowering blood glucose levels in a diabetic patient in need thereof comprising orally administering to the diabetic patient a therapeutically effective amount of a pharmaceutical composition prior to a surgical procedure and after a surgical procedure to lower blood glucose levels; wherein the pharmaceutical composition comprises from about 0.01 wt % to about 10 wt % of tranexamic acid, from about 1 wt % to about 10 wt % of polyethylene glycol, from about 1 wt % to about 10 wt % of glucose, and from about 0 wt % to about 10 wt % of electrolytes. In embodiments, the methods for lowering blood glucose are methods for reducing post-surgical blood glucose levels. In embodiments, the composition is administered as one or two doses prior to the surgical procedure. In embodiments, the composition is administered for one to ten days after the surgical procedure; or from one to nine days after the surgical procedure; or from one to eight days after the surgical procedure; or from one to seven days after the surgical procedure; or from one to six days after the surgical procedure; or from one to five days after the surgical procedure. In embodiments, the composition is administered for one to four days after the surgical procedure. In embodiments, the composition is administered for one to three days after the surgical procedure. In embodiments, the composition is administered for one to two days after the surgical procedure. When administered after the surgical procedure, the compositions may be administered by any means known in the art. For example, the compositions may be administered in one, two, or multiple doses over the course of the day, or may be administered via a nasogastric/orogastric tube over the course of the day (e.g., administered as a drip over a period of time, such as 8 to 24 hours). In embodiments, the methods further comprise administering the composition to the patient during the surgical procedure. In embodiments, the composition is orally administered as a liquid dosage form or a solid dosage form. In embodiments, the pharmaceutical compositions are in the form of an aqueous solution. In embodiments, the pharmaceutical comprises from about 0.1 wt % to about 5 wt % of tranexamic acid, from about 2 wt % to about 8 wt % of polyethylene glycol, from about 2 wt % to about 8 wt % of glucose, and from about 0.001 wt % to about 8 wt % of electrolytes. In embodiments, the pharmaceutical comprises from about 0.1 wt % to about 3 wt % of tranexamic acid, from about 2 wt % to about 6 wt % of polyethylene glycol, from about 3 wt % to about 7 wt % of glucose, and from about 0.01 wt % to about 3 wt % of electrolytes. In embodiments, the pharmaceutical comprises from about 0.3 wt % to about 2.0 wt % of tranexamic acid, from about 3.4 wt % to about 5.0 wt % of polyethylene glycol, from about 4.2 wt % to about 5.8 wt % of glucose, and from about 0.08 wt % to about 1.2 wt % of electrolytes. In embodiments the compositions comprise from about 0.4 wt % to about 1.9 wt % of tranexamic acid, from about 3.5 wt % to about 4.9 wt % of the polyethylene glycol, from about 4.3 wt % to about 5.7 wt % of the glucose, and from about 0.09 wt % to about 1.3 wt % of electrolytes. In embodiments the compositions comprise from about 0.5 wt % to about 1.8 wt % of tranexamic acid, from about 3.6 wt % to about 4.8 wt % of the polyethylene glycol, from about 4.4 wt % to about 5.6 wt % of the glucose, and from about 0.1 wt % to about 1.2 wt % of electrolytes. In embodiments the compositions comprise from about 0.6 wt % to about 1.6 wt % of tranexamic acid, from about 3.7 wt % to about 4.7 wt % of the polyethylene glycol, from about 4.5 wt % to about 5.5 wt % of the glucose, and from about 0.2 wt % to about 1.1 wt % of electrolytes. In embodiments the compositions comprise from about 0.7 wt % to about 1.5 wt % of tranexamic acid, from about 3.8 wt % to about 4.6 wt % of the polyethylene glycol, from about 4.6 wt % to about 5.4 wt % of the glucose, and from about 0.3 wt % to about 1.0 wt % of electrolytes. In embodiments the compositions comprise from about 0.8 wt % to about 1.4 wt % of tranexamic acid, from about 3.9 wt % to about 4.5 wt % of the polyethylene glycol, from about 4.7 wt % to about 5.3 wt % of the glucose, and from about 0.4 wt % to about 0.9 wt % of electrolytes. In embodiments the compositions comprise from about 0.9 wt % to about 1.3 wt % of tranexamic acid, and from about 4.0 wt % to about 4.4 wt % of the polyethylene glycol, from about 4.8 wt % to about 5.2 wt % of the glucose, and from about 0.5 wt % to about 0.8 wt % of electrolytes. In embodiments the compositions comprise from about 1.0 wt % to about 1.2 wt % of tranexamic acid, from about 4.1 wt % to about 4.3 wt % of the polyethylene glycol, from about 4.9 wt % to about 5.1 wt % of the glucose, and from about 0.6 wt % to about 0.7 wt % of electrolytes. In embodiments the compositions comprise about 1.1 wt % of tranexamic acid, about 4.2 wt % of the polyethylene glycol, about 5.0 wt % of the glucose, and about 0.67 wt % of electrolytes. In embodiments, the polyethylene glycol has an average molecular weight from about 1,000 Daltons to about 10,000 Daltons. In embodiments, the polyethylene glycol has an average molecular weight from about 2,000 Daltons to about 5,000 Daltons. In embodiments, the polyethylene glycol has an average molecular weight from about 3,000 Daltons to about 4,000 Daltons. In embodiments, the polyethylene glycol has an average molecular weight of about 3,500 Daltons. In embodiments, the electrolytes comprise sodium, potassium, calcium, magnesium, chloride, bicarbonate, phosphate, sulfate, or a combination of two or more thereof. In embodiments, the electrolytes comprise sodium sulfate, sodium bicarbonate, sodium chloride, potassium chloride, or a combination of two or more thereof. In embodiments, the electrolytes comprise sodium sulfate, sodium bicarbonate, sodium chloride, and potassium chloride.

In embodiments, the disclosure provides methods for lowering blood glucose levels in a diabetic patient in need thereof comprising orally administering to the diabetic patient a therapeutically effective amount of a pharmaceutical composition comprising from about 0.01 wt % to about 10 wt % of tranexamic acid, from about 1 wt % to about 10 wt % of polyethylene glycol, from about 1 wt % to about 10 wt % of glucose, and from about 0 wt % to about 10 wt % of electrolytes. In embodiments, the disclosure provides methods for treating diabetes in a patient in need thereof comprising orally administering to the patient a therapeutically effective amount of a pharmaceutical composition; wherein the pharmaceutical composition comprising from about 0.01 wt % to about 10 wt % of tranexamic acid, from about 1 wt % to about 10 wt % of polyethylene glycol, from about 1 wt % to about 10 wt % of glucose, and from about 0 wt % to about 10 wt % of electrolytes. In embodiments, the disclosure provides methods for improving glycemic control in diabetic patients in a patient in need thereof comprising orally administering to the patient a therapeutically effective amount of a pharmaceutical composition comprising from about 0.01 wt % to about 10 wt % of tranexamic acid, from about 1 wt % to about 10 wt % of polyethylene glycol, from about 1 wt % to about 10 wt % of glucose, and from about 0 wt % to about 10 wt % of electrolytes. The compositions may be administered by any means known in the art. For example, the compositions may be administered in one, two, or multiple doses over the course of the day, or may be administered via a nasogastric/orogastric tube over the course of the day (e.g., administered as a drip over a period of time, such as 8 to 24 hours). In embodiments, the composition is orally administered as a liquid dosage form or a solid dosage form. In embodiments, the pharmaceutical compositions are in the form of an aqueous solution. In embodiments, the pharmaceutical comprises from about 0.1 wt % to about 5 wt % of tranexamic acid, from about 2 wt % to about 8 wt % of polyethylene glycol, from about 2 wt % to about 8 wt % of glucose, and from about 0.001 wt % to about 8 wt % of electrolytes. In embodiments, the pharmaceutical comprises from about 0.1 wt % to about 3 wt % of tranexamic acid, from about 2 wt % to about 6 wt % of polyethylene glycol, from about 3 wt % to about 7 wt % of glucose, and from about 0.01 wt % to about 3 wt % of electrolytes. In embodiments, the pharmaceutical comprises from about 0.3 wt % to about 2.0 wt % of tranexamic acid, from about 3.4 wt % to about 5.0 wt % of polyethylene glycol, from about 4.2 wt % to about 5.8 wt % of glucose, and from about 0.08 wt % to about 1.2 wt % of electrolytes. In embodiments the compositions comprise from about 0.4 wt % to about 1.9 wt % of tranexamic acid, from about 3.5 wt % to about 4.9 wt % of the polyethylene glycol, from about 4.3 wt % to about 5.7 wt % of the glucose, and from about 0.09 wt % to about 1.3 wt % of electrolytes. In embodiments the compositions comprise from about 0.5 wt % to about 1.8 wt % of tranexamic acid, from about 3.6 wt % to about 4.8 wt % of the polyethylene glycol, from about 4.4 wt % to about 5.6 wt % of the glucose, and from about 0.1 wt % to about 1.2 wt % of electrolytes. In embodiments the compositions comprise from about 0.6 wt % to about 1.6 wt % of tranexamic acid, from about 3.7 wt % to about 4.7 wt % of the polyethylene glycol, from about 4.5 wt % to about 5.5 wt % of the glucose, and from about 0.2 wt % to about 1.1 wt % of electrolytes. In embodiments the compositions comprise from about 0.7 wt % to about 1.5 wt % of tranexamic acid, from about 3.8 wt % to about 4.6 wt % of the polyethylene glycol, from about 4.6 wt % to about 5.4 wt % of the glucose, and from about 0.3 wt % to about 1.0 wt % of electrolytes. In embodiments the compositions comprise from about 0.8 wt % to about 1.4 wt % of tranexamic acid, from about 3.9 wt % to about 4.5 wt % of the polyethylene glycol, from about 4.7 wt % to about 5.3 wt % of the glucose, and from about 0.4 wt % to about 0.9 wt % of electrolytes. In embodiments the compositions comprise from about 0.9 wt % to about 1.3 wt % of tranexamic acid, and from about 4.0 wt % to about 4.4 wt % of the polyethylene glycol, from about 4.8 wt % to about 5.2 wt % of the glucose, and from about 0.5 wt % to about 0.8 wt % of electrolytes. In embodiments the compositions comprise from about 1.0 wt % to about 1.2 wt % of tranexamic acid, from about 4.1 wt % to about 4.3 wt % of the polyethylene glycol, from about 4.9 wt % to about 5.1 wt % of the glucose, and from about 0.6 wt % to about 0.7 wt % of electrolytes. In embodiments the compositions comprise about 1.1 wt % of tranexamic acid, about 4.2 wt % of the polyethylene glycol, about 5.0 wt % of the glucose, and about 0.67 wt % of electrolytes. In embodiments, the polyethylene glycol has an average molecular weight from about 1,000 Daltons to about 10,000 Daltons. In embodiments, the polyethylene glycol has an average molecular weight from about 2,000 Daltons to about 5,000 Daltons. In embodiments, the polyethylene glycol has an average molecular weight from about 3,000 Daltons to about 4,000 Daltons. In embodiments, the polyethylene glycol has an average molecular weight of about 3,500 Daltons. In embodiments, the electrolytes comprise sodium, potassium, calcium, magnesium, chloride, bicarbonate, phosphate, sulfate, or a combination of two or more thereof. In embodiments, the electrolytes comprise sodium sulfate, sodium bicarbonate, sodium chloride, potassium chloride, or a combination of two or more thereof. In embodiments, the electrolytes comprise sodium sulfate, sodium bicarbonate, sodium chloride, and potassium chloride.

In embodiments, the methods described herein provide for lowering blood glucose levels in a diabetic patient by orally administering a therapeutically effective amount of a pharmaceutical composition comprising polyethylene glycol and, optionally (i) glucose, (ii) electrolytes, or (iii) glucose and electrolytes. In embodiments, the disclosure provides methods for treating diabetes in a patient in need thereof comprising orally administering to the patient a therapeutically effective amount of a pharmaceutical composition; wherein the pharmaceutical composition comprising polyethylene glycol and, optionally (i) glucose, (ii) electrolytes, or (iii) glucose and electrolytes. In embodiments, the disclosure provides methods for improving glycemic control in diabetic patients in a patient in need thereof comprising orally administering to the patient a therapeutically effective amount of a pharmaceutical composition; wherein the pharmaceutical composition comprises comprising polyethylene glycol and, optionally (i) glucose, (ii) electrolytes, or (iii) glucose and electrolytes. In embodiments, the pharmaceutical composition is administered (a) prior to the surgical procedure, (b) after the surgical procedure, or (c) prior to the surgical procedure and after the surgical procedure. The methods may further comprise administering the composition during the surgical procedure. In embodiments, the composition is administered to a diabetic patient who is not undergoing a surgical procedure or who is in a non-hospital setting. In embodiments, the composition is administered chronically. In embodiments, the polyethylene glycol has an average molecular weight from about 100 Daltons to about 30,000 Daltons. In embodiments, the polyethylene glycol has an average molecular weight from about 100 Daltons to about 25,000 Daltons. In embodiments, the polyethylene glycol has an average molecular weight from about 500 Daltons to about 20,000 Daltons. In embodiments, the polyethylene glycol has an average molecular weight from about 1,000 Daltons to about 15,000 Daltons. In embodiments, the polyethylene glycol has an average molecular weight from about 1,000 Daltons to about 10,000 Daltons. In embodiments, the polyethylene glycol has an average molecular weight from about 2,000 Daltons to about 5,000 Daltons. In embodiments, the polyethylene glycol has an average molecular weight from about 3,000 Daltons to about 4,000 Daltons. In embodiments, the polyethylene glycol has an average molecular weight of about 3,500 Daltons. In embodiments, the pharmaceutical compositions comprise from about 1 wt % to about 25 wt % of polyethylene glycol. In embodiments, the pharmaceutical compositions comprise from about 1 wt % to about 15 wt % of polyethylene glycol. In embodiments, the pharmaceutical compositions comprise from about 1 wt % to about 10 wt % of polyethylene glycol. In embodiments, the pharmaceutical compositions comprise from about 2 wt % to about 6 wt % of polyethylene glycol. In embodiments, the pharmaceutical compositions comprise from about 3 wt % to about 5 wt % of polyethylene glycol. In embodiments, the pharmaceutical compositions comprise from about 3.6 wt % to about 5.4 wt % of polyethylene glycol. In embodiments, the pharmaceutical compositions comprise from about 3.5 wt % to about 5.3 wt % of polyethylene glycol. In embodiments, the pharmaceutical compositions comprise from about 3.6 wt % to about 5.2 wt % of polyethylene glycol. In embodiments, the pharmaceutical compositions comprise from about 3.7 wt % to about 5.1 wt % of polyethylene glycol. In embodiments, the pharmaceutical compositions comprise from about 3.8 wt % to about 5.0 wt % of polyethylene glycol. In embodiments, the pharmaceutical compositions comprise from about 3.9 wt % to about 4.9 wt % of polyethylene glycol. In embodiments, the pharmaceutical compositions comprise from about 4.0 wt % to about 4.8 wt % of polyethylene glycol. In embodiments, the pharmaceutical compositions comprise from about 4.1 wt % to about 4.7 wt % of polyethylene glycol. In embodiments, the pharmaceutical compositions comprise from about 4.2 wt % to about 4.6 wt % of polyethylene glycol. In embodiments, the electrolytes comprise sodium, potassium, calcium, magnesium, chloride, bicarbonate, phosphate, sulfate, or a combination of two or more thereof. In embodiments, the electrolytes comprise sodium sulfate, sodium bicarbonate, sodium chloride, potassium chloride, or a combination of two or more thereof. In embodiments the compositions comprise from about 1 wt % to about 25 wt % of the polyethylene glycol, from about 1 wt % to about 25 wt % of the glucose, and, optionally, from about 0.001 wt % to about 25 wt % of electrolytes. In embodiments the compositions comprise from about 1 wt % to about 15 wt % of the polyethylene glycol, from about 1 wt % to about 15 wt % of the glucose, and, optionally, from about 0.05 wt % to about 20 wt % of electrolytes. In embodiments the compositions comprise from about 1 wt % to about 10 wt % of the polyethylene glycol, from about 1 wt % to about 10 wt % of the glucose, and, optionally, from about 0.05 wt % to about 5 wt % of electrolytes. In embodiments the compositions comprise from about 2 wt % to about 6 wt % of the polyethylene glycol, from about 2 wt % to about 6 wt % of the glucose, and, optionally, from about 0.1 wt % to about 2 wt % of electrolytes. In embodiments the compositions comprise from about 3.5 wt % to about 5.3 wt % of the polyethylene glycol, from about 3.3 wt % to about 5.7 wt % of the glucose, and, optionally, from about 0.09 wt % to about 1.6 wt % of electrolytes. In embodiments the compositions comprise from about 3.6 wt % to about 5.2 wt % of the polyethylene glycol, from about 3.4 wt % to about 5.6 wt % of the glucose, and, optionally, from about 0.1 wt % to about 1.5 wt % of electrolytes. In embodiments the compositions comprise from about 3.7 wt % to about 5.1 wt % of the polyethylene glycol, from about 3.5 wt % to about 5.5 wt % of the glucose, and, optionally, from about 0.2 wt % to about 1.4 wt % of electrolytes. In embodiments the compositions comprise from about 3.8 wt % to about 5.0 wt % of the polyethylene glycol, from about 3.6 wt % to about 5.4 wt % of the glucose, and, optionally, from about 0.3 wt % to about 1.3 wt % of electrolytes. In embodiments the compositions comprise from about 3.9 wt % to about 4.9 wt % of the polyethylene glycol, from about 3.7 wt % to about 5.3 wt % of the glucose, and, optionally, from about 0.4 wt % to about 1.2 wt % of electrolytes. In embodiments the compositions comprise from about 4.0 wt % to about 4.8 wt % of the polyethylene glycol, from about 3.8 wt % to about 5.2 wt % of the glucose, and, optionally, from about 0.5 wt % to about 1.1 wt % of electrolytes. In embodiments the compositions comprise from about 4.1 wt % to about 4.7 wt % of the polyethylene glycol, from about 3.9 wt % to about 5.1 wt % of the glucose, and, optionally, from about 0.6 wt % to about 1.0 wt % of electrolytes. In embodiments the compositions comprise from about 4.2 wt % to about 4.6 wt % of the polyethylene glycol, from about 4.0 wt % to about 5.0 wt % of the glucose, and, optionally, from about 0.67 wt % to about 0.96 wt % of electrolytes. In embodiments the compositions comprise about 4.6 wt % of the polyethylene glycol, about 4.0 wt % of the glucose, and, optionally, about 0.96 wt % of electrolytes. In embodiments the compositions comprise about 4.2 wt % of the polyethylene glycol, about 5.0 wt % of the glucose, and, optionally, about 0.67 wt % of electrolytes. In embodiments, the composition is orally administered as a liquid dosage form or a solid dosage form.

In embodiments, the methods described herein provide for lowering blood glucose levels in a diabetic patient by orally administering a therapeutically effective amount of a pharmaceutical composition comprising 1.1 wt % of tranexamic acid, about 4.6 wt % of polyethylene glycol, about 4.0 wt % of glucose, and about 0.96 wt % of at least one electrolyte. In embodiments, the disclosure provides methods for treating diabetes in a patient in need thereof comprising orally administering to the patient a therapeutically effective amount of a pharmaceutical composition comprising 1.1 wt % of tranexamic acid, about 4.6 wt % of polyethylene glycol, about 4.0 wt % of glucose, and about 0.96 wt % of at least one electrolyte. In embodiments, the disclosure provides methods for improving glycemic control in diabetic patients in a patient in need thereof comprising orally administering to the patient a therapeutically effective amount of a pharmaceutical composition comprising 1.1 wt % of tranexamic acid, about 4.6 wt % of polyethylene glycol, about 4.0 wt % of glucose, and about 0.96 wt % of at least one electrolyte. In embodiments, the pharmaceutical composition is administered (a) prior to the surgical procedure, (b) after the surgical procedure, or (c) prior to the surgical procedure and after the surgical procedure. In embodiments, the composition is administered to a diabetic patient who is not undergoing a surgical procedure or who is in a non-hospital setting. In embodiments, the composition is administered chronically. In embodiments, the composition is orally administered as a liquid dosage form or a solid dosage form. In embodiments, the pharmaceutical compositions are in the form of an aqueous solution. In embodiments, the pharmaceutical composition comprises about 0.9 wt % to about 1.3 wt % of tranexamic acid, about 4.4 wt % to about 4.8 wt % of polyethylene glycol, about 3.0 wt % to about 5.0 wt % of glucose, and about 0.9 wt % to about 1.0 wt % of at least one electrolyte. In embodiments, the pharmaceutical composition comprises about 0.7 wt % to about 1.5 wt % of tranexamic acid, about 4.2 wt % to about 5.0 wt % of polyethylene glycol, about 2.0 wt % to about 6.0 wt % of glucose, and about 0.8 wt % to about 1.1 wt % of at least one electrolyte. In embodiments, the pharmaceutical composition comprises about 0.4 wt % to about 1.8 wt % of tranexamic acid, about 3.9 wt % to about 5.3 wt % of polyethylene glycol, about 1.0 wt % to about 7.0 wt % of glucose, and about 0.6 wt % to about 1.3 wt % of at least one electrolyte. In embodiments, the pharmaceutical composition comprises about 0.1 wt % to about 5.0 wt % of tranexamic acid, about 0.1 wt % to about 10 wt % of polyethylene glycol, about 0.1 wt % to about 20.0 wt % of glucose, and about 0.05 wt % to about 10 wt % of at least one electrolyte.

In embodiments, the methods described herein provide for lowering blood glucose levels in a diabetic patient by orally administering a therapeutically effective amount of a pharmaceutical composition comprising about 0.9 wt % to about 1.3 wt % of tranexamic acid, about 4.4 wt % to about 4.8 wt % of polyethylene glycol, and about 0.9 wt % to about 1.0 wt % of at least one electrolyte. In embodiments, the disclosure provides methods for treating diabetes in a patient in need thereof comprising orally administering to the patient a therapeutically effective amount of a pharmaceutical composition comprising about 0.9 wt % to about 1.3 wt % of tranexamic acid, about 4.4 wt % to about 4.8 wt % of polyethylene glycol, and about 0.9 wt % to about 1.0 wt % of at least one electrolyte. In embodiments, the disclosure provides methods for improving glycemic control in diabetic patients in a patient in need thereof comprising orally administering to the patient a therapeutically effective amount of a pharmaceutical composition comprising about 0.9 wt % to about 1.3 wt % of tranexamic acid, about 4.4 wt % to about 4.8 wt % of polyethylene glycol, and about 0.9 wt % to about 1.0 wt % of at least one electrolyte. In embodiments, the pharmaceutical composition is administered (a) prior to a surgical procedure, (b) after a surgical procedure, or (c) prior to a surgical procedure and after a surgical procedure. In embodiments, the composition is administered to a diabetic patient who is not undergoing a surgical procedure or who is in a non-hospital setting. In embodiments, the composition is administered chronically. In embodiments, the composition is orally administered as a liquid dosage form or a solid dosage form. In embodiments, the pharmaceutical compositions are in the form of an aqueous solution. In embodiments, the pharmaceutical composition comprises about 0.7 wt % to about 1.5 wt % of tranexamic acid, about 4.2 wt % to about 5.0 wt % of polyethylene glycol, and about 0.8 wt % to about 1.1 wt % of at least one electrolyte. In embodiments, the pharmaceutical composition comprises about 0.4 wt % to about 1.8 wt % of tranexamic acid, about 3.9 wt % to about 5.3 wt % of polyethylene glycol, and about 0.6 wt % to about 1.3 wt % of at least one electrolyte. In embodiments, the pharmaceutical composition comprises about 0.1 wt % to about 5.0 wt % of tranexamic acid, about 0.1 wt % to about 10 wt % of polyethylene glycol, and about 0.05 wt % to about 10 wt % of at least one electrolyte.

In embodiments, the methods described herein provide for lowering blood glucose levels in a diabetic patient by orally administering a therapeutically effective amount of a pharmaceutical composition comprising about 0.9 wt % to about 1.3 wt % of tranexamic acid, about 3.0 wt % to about 5.0 wt % of glucose, and about 0.9 wt % to about 1.0 wt % of at least one electrolyte. In embodiments, the disclosure provides methods for treating diabetes in a patient in need thereof comprising orally administering to the patient a therapeutically effective amount of a pharmaceutical composition comprising about 0.9 wt % to about 1.3 wt % of tranexamic acid, about 3.0 wt % to about 5.0 wt % of glucose, and about 0.9 wt % to about 1.0 wt % of at least one electrolyte. In embodiments, the disclosure provides methods for improving glycemic control in diabetic patients in a patient in need thereof comprising orally administering to the patient a therapeutically effective amount of a pharmaceutical composition comprising about 0.9 wt % to about 1.3 wt % of tranexamic acid, about 3.0 wt % to about 5.0 wt % of glucose, and about 0.9 wt % to about 1.0 wt % of at least one electrolyte. In embodiments, the pharmaceutical composition is administered (a) prior to a surgical procedure, (b) after a surgical procedure, or (c) prior to a surgical procedure and after a surgical procedure. In embodiments, the composition is administered to a diabetic patient who is not undergoing a surgical procedure or who is in a non-hospital setting. In embodiments, the composition is administered chronically. In embodiments, the composition is orally administered as a liquid dosage form or a solid dosage form. In embodiments, the pharmaceutical compositions are in the form of an aqueous solution. In embodiments, the pharmaceutical composition comprises about 0.7 wt % to about 1.5 wt % of tranexamic acid, about 2.0 wt % to about 6.0 wt % of glucose, and about 0.8 wt % to about 1.1 wt % of at least one electrolyte. In embodiments, the pharmaceutical composition comprises about 0.4 wt % to about 1.8 wt % of tranexamic acid, about 1.0 wt % to about 7.0 wt % of glucose, and about 0.6 wt % to about 1.3 wt % of at least one electrolyte. In embodiments, the pharmaceutical composition comprises about 0.1 wt % to about 5.0 wt % of tranexamic acid, about 0.1 wt % to about 20.0 wt % of glucose, and about 0.05 wt % to about 10 wt % of at least one electrolyte.

In embodiments, the methods provide for lowering blood glucose levels in a diabetic patient by orally administering a therapeutically effective amount of a pharmaceutical composition comprising about 1.1 wt % of tranexamic acid, about 4.2 wt % of polyethylene glycol having an average molecular weight of about 3,350, about 5.0 wt % of glucose, and one or more electrolytes. In embodiments, the disclosure provides methods for treating diabetes in a patient in need thereof comprising orally administering to the patient a therapeutically effective amount of a pharmaceutical composition comprising about 1.1 wt % of tranexamic acid, about 4.2 wt % of polyethylene glycol having an average molecular weight of about 3,350, about 5.0 wt % of glucose, and one or more electrolytes. In embodiments, the disclosure provides methods for improving glycemic control in diabetic patients in a patient in need thereof comprising orally administering to the patient a therapeutically effective amount of a pharmaceutical composition comprising about 1.1 wt % of tranexamic acid, about 4.2 wt % of polyethylene glycol having an average molecular weight of about 3,350, about 5.0 wt % of glucose, and one or more electrolytes. In embodiments, the pharmaceutical composition is administered (a) prior to a surgical procedure, (b) after a surgical procedure, or (c) prior to a surgical procedure and after a surgical procedure. In embodiments, the composition is administered to a diabetic patient who is not undergoing a surgical procedure or who is in a non-hospital setting. In embodiments, the composition is administered chronically. In embodiments, the composition is orally administered as a liquid dosage form or a solid dosage form. In embodiments, the pharmaceutical compositions are in the form of an aqueous solution. In embodiments, the pharmaceutical composition comprises about 1.1 wt % of tranexamic acid, about 4.2 wt % of polyethylene glycol having an average molecular weight of about 3,350, about 5.0 wt % of glucose, about 0.4 wt % of sodium sulfate, about 0.12 wt % of sodium bicarbonate, about 0.1 wt % of sodium chloride, and about 0.05 wt % of potassium chloride.

In embodiments, the methods described herein provide for lowering blood glucose levels in a diabetic patient by orally administering a therapeutically effective amount of an pharmaceutical composition comprising about 1.0 wt % to about 1.2 wt % of tranexamic acid, about 4.1 wt % to about 4.3 wt % of polyethylene glycol having an average molecular weight of about 3,350, about 4.9 wt % to about 5.1 wt % of glucose, about 0.3 wt % to about 0.5 wt % of sodium sulfate, about 0.11 wt % to about 0.13 wt % of sodium bicarbonate, about 0.9 wt % to about 0.2 wt % of sodium chloride, and about 0.04 wt % to about 0.06 wt % of potassium chloride. In embodiments, the disclosure provides methods for treating diabetes in a patient in need thereof comprising orally administering to the patient a therapeutically effective amount of a pharmaceutical composition comprising about 1.0 wt % to about 1.2 wt % of tranexamic acid, about 4.1 wt % to about 4.3 wt % of polyethylene glycol having an average molecular weight of about 3,350, about 4.9 wt % to about 5.1 wt % of glucose, about 0.3 wt % to about 0.5 wt % of sodium sulfate, about 0.11 wt % to about 0.13 wt % of sodium bicarbonate, about 0.9 wt % to about 0.2 wt % of sodium chloride, and about 0.04 wt % to about 0.06 wt % of potassium chloride. In embodiments, the disclosure provides methods for improving glycemic control in diabetic patients in a patient in need thereof comprising orally administering to the patient a therapeutically effective amount of a pharmaceutical composition comprising about 1.0 wt % to about 1.2 wt % of tranexamic acid, about 4.1 wt % to about 4.3 wt % of polyethylene glycol having an average molecular weight of about 3,350, about 4.9 wt % to about 5.1 wt % of glucose, about 0.3 wt % to about 0.5 wt % of sodium sulfate, about 0.11 wt % to about 0.13 wt % of sodium bicarbonate, about 0.9 wt % to about 0.2 wt % of sodium chloride, and about 0.04 wt % to about 0.06 wt % of potassium chloride. In embodiments, the pharmaceutical composition is administered (a) prior to the surgical procedure, (b) after the surgical procedure, or (c) prior to the surgical procedure and after the surgical procedure. In embodiments, the composition is administered to a diabetic patient who is not undergoing a surgical procedure or who is in a non-hospital setting. In embodiments, the composition is administered chronically. In embodiments, the composition is orally administered as a liquid dosage form or a solid dosage form. In embodiments, the pharmaceutical compositions are in the form of an aqueous solution. In embodiments, the pharmaceutical composition comprises about 0.9 wt % to about 1.3 wt % of tranexamic acid, about 4.0 wt % to about 4.4 wt % of polyethylene glycol having an average molecular weight of about 3,350, about 4.8 wt % to about 5.2 wt % of glucose, about 0.2 wt % to about 0.6 wt % of sodium sulfate, about 0.10 wt % to about 0.14 wt % of sodium bicarbonate, about 0.8 wt % to about 0.3 wt % of sodium chloride, and about 0.03 wt % to about 0.07 wt % of potassium chloride. In embodiments, the pharmaceutical composition comprises about 0.8 wt % to about 1.4 wt % of tranexamic acid, about 3.9 wt % to about 4.5 wt % of polyethylene glycol having an average molecular weight of about 3,350, about 4.7 wt % to about 5.3 wt % of glucose, about 0.1 wt % to about 0.7 wt % of sodium sulfate, about 0.09 wt % to about 0.13 wt % of sodium bicarbonate, about 0.7 wt % to about 0.4 wt % of sodium chloride, and about 0.02 wt % to about 0.08 wt % of potassium chloride. In embodiments, the pharmaceutical composition comprises about 0.5 wt % to about 2 wt % of tranexamic acid, about 3 wt % to about 6 wt % of polyethylene glycol, about 3 wt % to about 7 wt % of glucose, about 0.01 wt % to about 2 wt % of sodium sulfate, about 0.01 wt % to about 1 wt % of sodium bicarbonate, about 0.01 wt % to about 1 wt % of sodium chloride, and about 0.01 wt % to about 1 wt % of potassium chloride.

In embodiments, the methods described herein provide for lowering blood glucose levels in a diabetic patient by orally administering a therapeutically effective amount of a pharmaceutical composition comprising about 1.0 wt % to about 1.2 wt % of tranexamic acid, about 4.1 wt % to about 4.3 wt % of polyethylene glycol, about 4.9 wt % to about 5.1 wt % of glucose, and one or more electrolytes. In embodiments, the disclosure provides methods for treating diabetes in a patient in need thereof comprising orally administering to the patient a therapeutically effective amount of a pharmaceutical composition comprising about 1.0 wt % to about 1.2 wt % of tranexamic acid, about 4.1 wt % to about 4.3 wt % of polyethylene glycol, about 4.9 wt % to about 5.1 wt % of glucose, and one or more electrolytes. In embodiments, the disclosure provides methods for improving glycemic control in diabetic patients in a patient in need thereof comprising orally administering to the patient a therapeutically effective amount of a pharmaceutical composition comprising about 1.0 wt % to about 1.2 wt % of tranexamic acid, about 4.1 wt % to about 4.3 wt % of polyethylene glycol, about 4.9 wt % to about 5.1 wt % of glucose, and one or more electrolytes. In embodiments, the pharmaceutical composition is administered (a) prior to the surgical procedure, (b) after the surgical procedure, or (c) prior to the surgical procedure and after the surgical procedure. In embodiments, the composition is administered to a diabetic patient who is not undergoing a surgical procedure or who is in a non-hospital setting. In embodiments, the composition is administered chronically. In embodiments, the composition is orally administered as a liquid dosage form or a solid dosage form. In embodiments, the pharmaceutical compositions are in the form of an aqueous solution. In embodiments, the pharmaceutical composition comprises about 0.9 wt % to about 1.3 wt % of tranexamic acid, about 4.0 wt % to about 4.4 wt % of polyethylene glycol, about 4.8 wt % to about 5.2 wt % of glucose, and one or more electrolytes. In embodiments, the pharmaceutical composition comprises about 0.8 wt % to about 1.4 wt % of tranexamic acid, about 3.9 wt % to about 4.5 wt % of polyethylene glycol, about 4.7 wt % to about 5.3 wt % of glucose, and one or more electrolytes.

In embodiments, the methods described herein provide for lowering blood glucose levels in a diabetic patient by orally administering a therapeutically effective amount of a pharmaceutical composition comprising one or more compositions described in U.S. Pat. Nos. 9,504,736, 9,314,442, or 9,775,821, the disclosures of which are incorporated by reference herein in its entirety. In embodiments, the pharmaceutical composition is administered (a) prior to the surgical procedure, (b) after the surgical procedure, or (c) prior to the surgical procedure and after the surgical procedure. In embodiments, the composition is administered to a diabetic patient who is not undergoing a surgical procedure or who is in a non-hospital setting. In embodiments, the composition is administered chronically. In embodiments, the composition is orally administered as a liquid dosage form or a solid dosage form. In embodiments, the pharmaceutical compositions are in the form of an aqueous solution.

In embodiments, the electrolyte in any of the pharmaceutical compositions described herein is sodium, potassium, calcium, magnesium, chloride, bicarbonate, phosphate, sulfate, or a combination of two or more thereof. In embodiments, the electrolyte is sodium, sulfate, bicarbonate, chloride, potassium, or a combination of two or more thereof In embodiments, the electrolyte is sodium sulfate, sodium bicarbonate, sodium chloride, potassium chloride, or a combination of two or more thereof. In embodiments, the electrolyte is sodium sulfate, sodium bicarbonate, sodium chloride, and potassium chloride. In embodiments, the pharmaceutical composition comprises electrolytes in an amount of about 0.4 wt % to about 0.57 wt % of sodium sulfate, about 0.12 wt % to about 0.17 wt % of sodium bicarbonate, about 0.1 wt % to about 0.1 wt % of sodium chloride, and about 0.05 wt % to about 0.07 wt % of potassium chloride. In embodiments, the pharmaceutical composition comprises electrolytes in an amount of about 0.3 wt % to about 0.6 wt % of sodium sulfate, about 0.11 wt % to about 0.18 wt % of sodium bicarbonate, about 0.09 wt % to about 0.2 wt % of sodium chloride, and about 0.04 wt % to about 0.08 wt % of potassium chloride. In embodiments, the pharmaceutical composition comprises electrolytes in an amount of about 0.1 wt % to about 1.0 wt % of sodium sulfate, about 0.05 wt % to about 1.0 wt % of sodium bicarbonate, about 0.01 wt % to about 1.0 wt % of sodium chloride, and about 0.01 wt % to about 1.0 wt % of potassium chloride. In embodiments, the pharmaceutical composition comprises electrolytes in an amount of about 0.57 wt % of sodium sulfate, about 0.17 wt % of sodium bicarbonate, about 0.15 wt % of sodium chloride, and about 0.07 wt % of potassium chloride. In embodiments, the pharmaceutical composition comprises electrolytes in an amount of about 0.5 wt % to about 0.6 wt % of sodium sulfate, about 0.1 wt % to about 0.2 wt % of sodium bicarbonate, about 0.1 wt % to about 0.2 wt % of sodium chloride, and about 0.06 wt % to about 0.09 wt % of potassium chloride. In embodiments, the pharmaceutical composition comprises electrolytes in an amount of about 0.1 wt % to about 1.0 wt % of sodium sulfate, about 0.01 wt % to about 1.0 wt % of sodium bicarbonate, about 0.01 wt % to about 1.0 wt % of sodium chloride, and about 0.01 wt % to about 1.0 wt % of potassium chloride. In embodiments, the pharmaceutical composition comprises electrolytes in an amount of about 0.4 wt % of sodium sulfate, about 0.12 wt % of sodium bicarbonate, about 0.1 wt % of sodium chloride, and about 0.05 wt % of potassium chloride.

In embodiments, the polyethylene glycol used in any of the pharmaceutical compositions described herein has an average molecular weight from about 100 Daltons to about 50,000 Daltons; or from about 100 Daltons to about 40,000 Daltons; or from about 100 Daltons to about 30,000 Daltons; or from about 100 Daltons to about 25,000 Daltons; or from about 100 Daltons to about 20,000 Daltons; or from about 100 Daltons to about 15,000 Daltons; from about 100 Daltons to about 10,000 Daltons; or from about 100 Daltons to about 9,000 Daltons; or from about 500 Daltons to about 8,000 Daltons; or from about 1,000 Daltons to about 6,000 Daltons; or from about 2,000 Daltons to about 5,000 Daltons; or from about 2,500 Daltons to about 4,500 Daltons; or from about 3,000 Daltons to about 4,000 Daltons; or from about 3,000 Daltons to about 3,500 Daltons; or from about 3,300 Daltons to about 3,400 Daltons. In embodiments, the polyethylene glycol has an average molecular weight of about 3,000 Daltons to about 4,500 Daltons. In embodiments, the polyethylene glycol has an average molecular weight of about 4,000 Daltons. In embodiments, the polyethylene glycol has an average molecular weight of about 3,350 Daltons. In embodiments, the polyethylene glycol has an average molecular weight of about 3,000 Daltons. In embodiments, the polyethylene glycol has an average molecular weight of about 3,500 Daltons.

The disclosure provides a pharmaceutical composition of Formulation B shown in Table 2. This pharmaceutical composition comprises about 1.1 wt % of tranexamic acid, about 4.2 wt % of polyethylene glycol having an average molecular weight of about 3,350, about 5.0 wt % of glucose, and one or more electrolytes. In embodiments, the pharmaceutical composition comprises about 1.1 wt % of tranexamic acid, about 4.2 wt % of polyethylene glycol having an average molecular weight of about 3,350, about 5.0 wt % of glucose, about 0.4 wt % of sodium sulfate, about 0.12 wt % of sodium bicarbonate, about 0.1 wt % of sodium chloride, and about 0.05 wt % of potassium chloride.

The disclosure provides a pharmaceutical composition comprising about 1.0 wt % to about 1.2 wt % of tranexamic acid, about 4.1 wt % to about 4.3 wt % of polyethylene glycol having an average molecular weight of about 3,350, about 4.9 wt % to about 5.1 wt % of glucose, about 0.3 wt % to about 0.5 wt % of sodium sulfate, about 0.11 wt % to about 0.13 wt % of sodium bicarbonate, about 0.9 wt % to about 0.2 wt % of sodium chloride, and about 0.04 wt % to about 0.06 wt % of potassium chloride. In embodiments, the pharmaceutical composition comprises about 0.9 wt % to about 1.3 wt % of tranexamic acid, about 4.0 wt % to about 4.4 wt % of polyethylene glycol having an average molecular weight of about 3,350, about 4.8 wt % to about 5.2 wt % of glucose, about 0.2 wt % to about 0.6 wt % of sodium sulfate, about 0.10 wt % to about 0.14 wt % of sodium bicarbonate, about 0.8 wt % to about 0.3 wt % of sodium chloride, and about 0.03 wt % to about 0.07 wt % of potassium chloride. In embodiments, the pharmaceutical composition comprises about 0.8 wt % to about 1.4 wt % of tranexamic acid, about 3.9 wt % to about 4.5 wt % of polyethylene glycol having an average molecular weight of about 3,350, about 4.7 wt % to about 5.3 wt % of glucose, about 0.1 wt % to about 0.7 wt % of sodium sulfate, about 0.09 wt % to about 0.13 wt % of sodium bicarbonate, about 0.7 wt % to about 0.4 wt % of sodium chloride, and about 0.02 wt % to about 0.08 wt % of potassium chloride. In embodiments, the pharmaceutical composition comprises about 0.5 wt % to about 2 wt % of tranexamic acid, about 3 wt % to about 6 wt % of polyethylene glycol, about 3 wt % to about 7 wt % of glucose, about 0.01 wt % to about 2 wt % of sodium sulfate, about 0.01 wt % to about 1 wt % of sodium bicarbonate, about 0.01 wt % to about 1 wt % of sodium chloride, and about 0.01 wt % to about 1 wt % of potassium chloride.

The disclosure provides a pharmaceutical composition comprising about 1.0 wt % to about 1.2 wt % of tranexamic acid, about 4.1 wt % to about 4.3 wt % of polyethylene glycol, about 4.9 wt % to about 5.1 wt % of glucose, and one or more electrolytes. In embodiments, the pharmaceutical composition comprises about 0.9 wt % to about 1.3 wt % of tranexamic acid, about 4.0 wt % to about 4.4 wt % of polyethylene glycol, about 4.8 wt % to about 5.2 wt % of glucose, and one or more electrolytes. In embodiments, the pharmaceutical composition comprises about 0.8 wt % to about 1.4 wt % of tranexamic acid, about 3.9 wt % to about 4.5 wt % of polyethylene glycol, about 4.7 wt % to about 5.3 wt % of glucose, and one or more electrolytes.

In embodiments, the pharmaceutical compositions described herein have a volume of about 100 ml to about 2,500 ml. In embodiments, the pharmaceutical compositions have a volume of about 250 ml to about 1,000 ml. In embodiments, the pharmaceutical compositions have a volume of about 400 ml to about 1,000 ml. In embodiments, the pharmaceutical compositions have a volume of about 600 ml to about 800 ml. In embodiments, the pharmaceutical compositions have a volume of about 700 ml. In embodiments, the pharmaceutical compositions have a volume of about 500 ml. In embodiments, the pharmaceutical compositions have a volume of about 1,000 ml. In embodiments, the pharmaceutical compositions have a volume of about 750 ml.

In embodiments, the pharmaceutical compositions described herein have a volume of about 250 ml to about 1,000 ml, and comprise about 1.0 gram to about 10.0 grams tranexamic acid, about 5.0 grams to about 40.0 grams polyethylene glycol having a molecular weight of about 3,350, about 5 grams to about 40 grams of glucose, and about 0.01 grams to about 15.0 grams of at least one electrolyte. In embodiments, the pharmaceutical compositions described herein have a volume of about 400 ml to about 1,000 ml, and comprise about 5.0 grams to about 10.0 grams tranexamic acid, about 25.0 grams to about 40.0 grams polyethylene glycol having a molecular weight of about 3,350, about 15 grams to about 40 grams of glucose, and about 1 gram to about 15.0 grams of at least one electrolyte. In embodiments, the pharmaceutical compositions described herein have a volume of about 600 ml to about 800 ml, and comprise about 6.0 grams to about 9.0 grams tranexamic acid, about 28.0 grams to about 37.0 grams polyethylene glycol having a molecular weight of about 3,350, about 22 grams to about 34 grams of glucose, and about 4 grams to about 10.0 grams of at least one electrolyte. In embodiments, the pharmaceutical compositions described herein have a volume of about 600 ml to about 800 ml, and comprise about 6.5 grams to about 8.5 grams tranexamic acid, about 30.0 grams to about 35.0 grams polyethylene glycol having a molecular weight of about 3,350, about 25 grams to about 31 grams of glucose, about 3 grams to about 5 grams of sodium sulfate, about 0.8 grams to about 1.6 grams sodium bicarbonate, about 0.5 grams to about 1.5 grams sodium chloride, and about 0.1 grams to about 1.0 grams potassium chloride. In embodiments, the pharmaceutical compositions described herein have a volume of about 700 ml and comprise about 7.5 grams tranexamic acid, about 32.5 grams polyethylene glycol having a molecular weight of about 3,350, about 28 grams of glucose, about 4 grams of sodium sulfate, about 1.2 grams sodium bicarbonate, about 1.0 grams sodium chloride, and about 0.5 grams potassium chloride. In embodiments, the pharmaceutical compositions described herein have a volume of about 700 ml and comprise about 7.5 grams tranexamic acid, about 29.5 grams polyethylene glycol having a molecular weight of about 3,350, about 35 grams of glucose, about 2.8 grams of sodium sulfate, about 0.85 grams sodium bicarbonate, about 0.7 grams sodium chloride, and about 0.35 grams potassium chloride.

In embodiments, the pharmaceutical compositions described herein have a volume of about 250 ml to about 1,000 ml, and comprise about 1.0 gram to about 10.0 grams tranexamic acid, about 5.0 grams to about 40.0 grams polyethylene glycol having a molecular weight of about 3,350, and about 0.01 grams to about 15.0 grams of at least one electrolyte. In embodiments, the pharmaceutical compositions described herein have a volume of about 400 ml to about 1,000 ml, and comprise about 5.0 grams to about 10.0 grams tranexamic acid, about 25.0 grams to about 40.0 grams polyethylene glycol having a molecular weight of about 3,350, and about 1 gram to about 15.0 grams of at least one electrolyte. In embodiments, the pharmaceutical compositions described herein have a volume of about 600 ml to about 800 ml, and comprise about 6.0 grams to about 9.0 grams tranexamic acid, about 28.0 grams to about 37.0 grams polyethylene glycol having a molecular weight of about 3,350, and about 4 grams to about 10.0 grams of at least one electrolyte. In embodiments, the pharmaceutical compositions described herein have a volume of about 600 ml to about 800 ml, and comprise about 6.5 grams to about 8.5 grams tranexamic acid, about 30.0 grams to about 35.0 grams polyethylene glycol having a molecular weight of about 3,350, about 3 grams to about 5 grams of sodium sulfate, about 0.8 grams to about 1.6 grams sodium bicarbonate, about 0.5 grams to about 1.5 grams sodium chloride, and about 0.1 grams to about 1.0 grams potassium chloride. In embodiments, the pharmaceutical compositions described herein have a volume of about 700 ml and comprise about 7.5 grams tranexamic acid, about 32.5 grams polyethylene glycol having a molecular weight of about 3,350, about 4 grams of sodium sulfate, about 1.2 grams sodium bicarbonate, about 1.0 grams sodium chloride, and about 0.5 grams potassium chloride.

In embodiments, the pharmaceutical composition is Formulation A, set forth in Table 1.

TABLE 1
Formulation A

| | | |
|---|---|---|
| | Formulation A | Formulation A |
| Component | (in grams) | (in wt %) |

| | | | | |
|---|---|---|---|---|
| Tranexamic acid | 7.5 | g | 1.1 | wt % |
| PEG 3350 | 32.5 | g | 4.6 | wt % |
| Glucose | 28 | g | 4.0 | wt % |
| Sodium Sulfate | 4 | g | 0.57 | wt % |
| Sodium Bicarbonate | 1.2 | g | 0.17 | wt % |
| Sodium Chloride | 1.0 | g | 0.15 | wt % |
| Potassium Chloride | 0.5 | g | 0.07 | wt % |

In embodiments, the pharmaceutical composition is Formulation B, set forth in Table 2.

TABLE 2
Formulation B

| | | |
|---|---|---|
| | Formulation B | Formulation B |
| Component | (in grams) | (in wt %) |

| | | | | |
|---|---|---|---|---|
| Tranexamic acid | 7.5 | g | 1.1 | wt % |
| PEG 3350 | 29.5 | g | 4.2 | wt % |
| Glucose | 35 | g | 5.0 | wt % |
| Sodium Sulfate | 2.8 | g | 0.4 | wt % |
| Sodium Bicarbonate | 0.85 | g | 0.12 | wt % |
| Sodium Chloride | 0.7 | g | 0.1 | wt % |
| Potassium Chloride | 0.35 | g | 0.05 | wt % |

In embodiments, the pharmaceutical composition is Formulation C, set forth in Table 3.

TABLE 3
Formulation C.

| | | |
|---|---|---|
| | Formulation C | Formulation C |
| Component | (in grams) | (in wt %) |

| | | | | |
|---|---|---|---|---|
| Tranexamic acid | 7.8 | g | 0.8 | wt % |
| PEG 3350 | 50.3 | g | 5.0 | wt % |
| Glucose | 40 | g | 4.0 | wt % |
| Sodium Sulfate | 5.7 | g | 0.57 | wt % |
| Sodium Bicarbonate | 1.7 | g | 0.17 | wt % |
| Sodium Chloride | 1.5 | g | 0.15 | wt % |
| Potassium Chloride | 0.7 | g | 0.07 | wt % |

The pharmaceutical compositions described herein can include a single agent (e.g., tranexamic acid) or more than one active agent. The compositions for administration will commonly include an agent as described herein dissolved in a pharmaceutically acceptable carrier, preferably an aqueous carrier. A variety of aqueous carriers can be used, e.g., water, buffered saline, saline, sterile saline, and the like. The compositions may contain other pharmaceutically acceptable excipients.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients can be used in the compositions described herein.

The term “dosage form” is the form in which the dose is to be administered to the subject or patient. The drug is generally administered as part of a formulation that includes nonmedical agents, i.e., pharmaceutically acceptable excipients. The dosage form has unique physical and pharmaceutical characteristics. Dosage forms, for example, may be solid or liquid. “Dosage forms” may include for example, a capsule, tablet, caplet, gel caplet (gel cap), syrup, a liquid composition, a powder, a concentrated powder, a concentrated powder admixed with a liquid, a chewable form, a swallowable form, a dissolvable form, an effervescent, a granulated form, and an oral liquid solution. In embodiments, the dosage form is a solid dosage form, and more specifically, comprises a tablet, capsule, or caplet. In specific embodiments, the dosage form in an aqueous dosage form, such as an aqueous pharmaceutical composition. The aqueous pharmaceutical compositions described herein can be presented in unit-dose or multi-dose sealed containers, such as ampules and vials. Thus, the composition can be in unit dosage form. In such form, the preparation is subdivided into unit doses containing appropriate quantities of the active component. Thus, the compositions can be administered in a variety of unit dosage forms depending upon the method of administration. In embodiments, the pharmaceutical compositions described herein are stored in two separate containers prior to administration, wherein one container comprises tranexamic acid, polyethylene glycol, and at least one electrolyte, and another container comprises glucose. Prior to oral or lavage administration, the contents of the containers are mixed together to form the pharmaceutical compositions described herein. In embodiments, the pharmaceutical compositions described herein are stored in a single container prior to administration to a patient.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art. For example, the pharmaceutical composition described herein can be administered as a single dose prior to a surgical procedure, as two doses prior to a surgical procedure, as three doses prior to a surgical procedure, or as four doses prior to a surgical procedure.

In embodiments, the pharmaceutical composition is administered as a single dose prior to a surgical procedure. When administered as a single dose, the composition may be orally administered from about 8 hours to about 5 minutes prior to a surgical procedure, or from about 6 hours to 5 minutes prior to a surgical procedure, or from about 4 hours to about 5 minutes prior to a surgical procedure, or from about 3 hours to about 5 minutes prior to a surgical procedure, or from about 2 hour to about 5 minutes prior to a surgical procedure, or about 1 hour to about 5 minutes prior to a surgical procedure. In embodiments, the single dose is an amount of about 100 ml to about 2,000 ml; or about 200 ml to about 1,500 ml; or about 300 ml to about 1,200 ml; or about 400 ml to about 1,000 ml; or about 500 ml to about 900 ml; or about 600 ml to about 800 ml; or about 700 ml.

In embodiments, the pharmaceutical composition is administered as two doses prior to a surgical procedure. When administered as two doses prior to a surgical procedure, a first dose is administered about 4.1 hours to about 12 hours prior to the surgical procedure, and a second dose is administered about 4 hours to about 5 minutes prior to the surgical procedure. In embodiments, a first dose is administered about 5 hours to about 10 hours prior to the surgical procedure, and a second dose is administered about 3 hours to about 5 minutes prior to the surgical procedure. In embodiments, a first dose is administered about 6 hours to about 8 hours prior to the surgical procedure, and a second dose is administered about 2 hours to about 5 minutes prior to the surgical procedure. In embodiments, a first dose is administered about 6 hours to about 8 hours prior to the surgical procedure, and a second dose is administered about 1 hour to about 5 minutes prior to the surgical procedure. In embodiments, the first dose is about 100 ml to about 600 ml, and the second dose is about 100 ml to about 600 ml. In embodiments, the first dose is about 200 ml to about 500 ml, and the second dose is about 200 ml to about 500 ml. In embodiments, the first dose is about 250 ml to about 450 ml, and the second dose is about 250 ml to about 450 ml. In embodiments, the first dose is about 300 ml to about 400 ml, and the second dose is about 300 ml to about 400 ml. In embodiments, the first dose is about 325 ml to about 375 ml, and the second dose is about 325 ml to about 375 ml. In embodiments, the first dose is about 350 ml, and the second dose is about 350 ml.

In embodiments, the first dose is administered after completion of pre-surgical bowel preparation, and the second dose is administered prior to the surgical procedure. In embodiments, the first dose is administered about 15 minutes to about 2 hours after completion of pre-surgical bowel preparation, and the second dose is administered about 3 hours to about 5 minutes prior to the surgical procedure. In embodiments, the first dose is administered about 30 minutes to about 2 hours after completion of pre-surgical bowel preparation, and the second dose is administered about 2 hours to about 5 minutes prior to the surgical procedure. In embodiments, the first dose is administered about 30 minutes to about 1 hour after completion of pre-surgical bowel preparation, and the second dose is administered about 1 hour to about 5 minutes prior to the surgical procedure. In embodiments, the first dose is about 100 ml to about 600 ml, and the second dose is about 100 ml to about 600 ml. In embodiments, the first dose is about 200 ml to about 500 ml, and the second dose is about 200 ml to about 500 ml. In embodiments, the first dose is about 250 ml to about 450 ml, and the second dose is about 250 ml to about 450 ml. In embodiments, the first dose is about 300 ml to about 400 ml, and the second dose is about 300 ml to about 400 ml. In embodiments, the first dose is about 325 ml to about 375 ml, and the second dose is about 325 ml to about 375 ml. In embodiments, the first dose is about 350 ml, and the second dose is about 350 ml. “Pre-surgical bowel preparation” refers to the cleansing or emptying of the bowels prior to surgery and is accomplished by methods known in the art.

In embodiments, the pharmaceutical composition is administered as two doses prior to a surgical procedure. When administered as two doses prior to a surgical procedure, a first dose is administered about 4.1 hours to about 12 hours prior to the surgical procedure, and a second dose is administered about 4 hours to about 5 minutes prior to the surgical procedure. In embodiments, a first dose is administered about 5 hours to about 10 hours prior to the surgical procedure, and a second dose is administered about 3 hours to about 5 minutes prior to the surgical procedure. In embodiments, a first dose is administered about 6 hours to about 8 hours prior to the surgical procedure, and a second dose is administered about 2 hours to about 5 minutes prior to the surgical procedure. In embodiments, a first dose is administered about 6 hours to about 8 hours prior to the surgical procedure, and a second dose is administered about 1 hour to about 5 minutes prior to the surgical procedure. In embodiments, the first dose is about 100 ml to about 600 ml, and the second dose is about 100 ml to about 600 ml. In embodiments, the first dose is about 200 ml to about 500 ml, and the second dose is about 200 ml to about 500 ml. In embodiments, the first dose is about 250 ml to about 450 ml, and the second dose is about 250 ml to about 450 ml. In embodiments, the first dose is about 300 ml to about 400 ml, and the second dose is about 300 ml to about 400 ml. In embodiments, the first dose is about 325 ml to about 375 ml, and the second dose is about 325 ml to about 375 ml. In embodiments, the first dose is about 350 ml, and the second dose is about 350 ml.

In embodiments, the first dose is administered after completion of pre-surgical bowel preparation, and the second dose is administered prior to the surgical procedure. In embodiments, the first dose is administered about 15 minutes to about 2 hours after completion of pre-surgical bowel preparation, and the second dose is administered about 3 hours to about 5 minutes prior to the surgical procedure. In embodiments, the first dose is administered about 30 minutes to about 2 hours after completion of pre-surgical bowel preparation, and the second dose is administered about 2 hours to about 5 minutes prior to the surgical procedure. In embodiments, the first dose is administered about 30 minutes to about 1 hour after completion of pre-surgical bowel preparation, and the second dose is administered about 1 hour to about 5 minutes prior to the surgical procedure. In embodiments, the first dose is about 100 ml to about 600 ml, and the second dose is about 100 ml to about 600 ml. In embodiments, the first dose is about 200 ml to about 500 ml, and the second dose is about 200 ml to about 500 ml. In embodiments, the first dose is about 250 ml to about 450 ml, and the second dose is about 250 ml to about 450 ml. In embodiments, the first dose is about 300 ml to about 400 ml, and the second dose is about 300 ml to about 400 ml. In embodiments, the first dose is about 325 ml to about 375 ml, and the second dose is about 325 ml to about 375 ml. In embodiments, the first dose is about 350 ml, and the second dose is about 350 ml. “Pre-surgical bowel preparation” refers to the cleansing or emptying of the bowels prior to surgery and is accomplished by methods known in the art.

The specification is also directed to the following numbered embodiments.

Embodiment 1. A method for lowering blood glucose levels in a diabetic patient in need thereof, the method comprising orally administering to the diabetic patient a therapeutically effective amount of an aqueous pharmaceutical composition prior to a surgical procedure to lower blood glucose levels; wherein the composition comprises an insulin receptor protease inhibitor.

Embodiment 2. A method for lowering blood glucose levels in a diabetic patient in need thereof, the method comprising orally administering to the diabetic patient a therapeutically effective amount of an aqueous pharmaceutical composition after a surgical procedure to lower blood glucose levels; wherein the composition comprises an insulin receptor protease inhibitor.

Embodiment 3. A method for lowering blood glucose levels in a diabetic patient in need thereof, the method comprising orally administering to the diabetic patient a therapeutically effective amount of an aqueous pharmaceutical composition prior to a surgical procedure and after a surgical procedure to lower blood glucose levels; wherein the composition comprises an insulin receptor protease inhibitor.

Embodiment 4. The method of any one of embodiments 1 to 3, further comprising administering a therapeutically effective amount of an aqueous pharmaceutical composition comprising an insulin receptor protease inhibitor during the surgical procedure.

Embodiment 5. The method of any one of embodiments 1 to 4, wherein the method for lowering blood glucose levels is a method for reducing post-surgical blood glucose levels.

Embodiment 6. The method of any one of embodiments 2 or 5, comprising administering the composition for at least two days after the surgical procedure.

Embodiment 7. The method of any one of embodiments 2 or 5, comprising administering the composition for at least three days after the surgical procedure.

Embodiment 8. The method of any one of embodiments 2 or 5, comprising administering the composition from one to four days after the surgical procedure.

Embodiment 9. The method of any one of embodiments 2 or 5, comprising administering the composition from one to ten days after the surgical procedure.

Embodiment 10. The method of any one of embodiments 1 to 9, wherein the insulin receptor protease inhibitor is a serine protease inhibitor, a matrix metalloproteinase inhibitor, or a lipase.

Embodiment 11. The method of any one of embodiments 1 to 9, wherein the insulin receptor protease inhibitor is a serine protease inhibitor.

Embodiment 12. The method of any one of embodiments 1 to 9, wherein the insulin receptor protease inhibitor is tranexamic acid, gabexate mesilate, camostat mesilate, nafamostat mesilate, α-1-anti-trypsin, α-2-macroglobulin, pancreatic lipase, gastric lipase, pancreatic lipase-related protein 1, pancreatic lipase-related protein 2, aprotinin, or a combination of two or more thereof.

Embodiment 13. The method of any one of embodiments 1 to 9, wherein the insulin receptor protease inhibitor is tranexamic acid.

Embodiment 14. The method of any one of embodiments 1 to 13, wherein the composition comprises from about of 0.1 wt % to about 25 wt % of the insulin receptor protease inhibitor

Embodiment 15. The method of any one of embodiments 1 to 13, wherein the composition comprises from about of 0.1 wt % to about 10 wt % of the insulin receptor protease inhibitor.

Embodiment 16. The method of any one of embodiments 1 to 13, wherein the composition comprises from about of 0.1 wt % to about 5 wt % of the insulin receptor protease inhibitor.

Embodiment 17. The method of any one of embodiments 1 to 13, wherein the composition comprises from about of 0.5 wt % to about 2 wt % of the insulin receptor protease inhibitor.

Embodiment 18. The method of any one of embodiments 1 to 17, wherein the composition further comprises polyethylene glycol.

Embodiment 19. The method of embodiment 18, wherein the polyethylene glycol has an average molecular weight from about 100 Daltons to about 50,000 Daltons.

Embodiment 20. The method of embodiment 18, wherein the polyethylene glycol has an average molecular weight from about 100 Daltons to about 30,000 Daltons.

Embodiment 21. The method of embodiment 18, wherein the polyethylene glycol has an average molecular weight from about 1,000 Daltons to about 10,000 Daltons.

Embodiment 22. The method of embodiment 18, wherein the polyethylene glycol has an average molecular weight of about 3,500 Daltons.

Embodiment 23. The method of any one of embodiments 18 to 22, wherein the composition comprises from about 1 wt % to about 25 wt % of polyethylene glycol.

Embodiment 24. The method of any one of embodiments 18 to 22, wherein the composition comprises from about 1 wt % to about 15 wt % of polyethylene glycol.

Embodiment 25. The method of any one of embodiments 18 to 22, wherein the composition comprises from about 1 wt % to about 10 wt % of polyethylene glycol.

Embodiment 26. The method of any one of embodiments 18 to 22, wherein the composition comprises from about 2 wt % to about 6 wt % of polyethylene glycol.

Embodiment 27. The method of any one of embodiment 1 to 26, wherein the composition further comprises glucose.

Embodiment 28. The method of embodiment 27, wherein the composition comprises from about 1 wt % to about 25 wt % of glucose.

Embodiment 29. The method of embodiment 27, wherein the composition comprises from about 1 wt % to about 15 wt % of glucose.

Embodiment 30. The method of embodiment 27, wherein the composition comprises from about 1 wt % to about 10 wt % of glucose.

Embodiment 31. The method of embodiment 27, wherein the composition comprises from about 2 wt % to about 6 wt % of glucose.

Embodiment 32. The method of any one of embodiments 1 to 31, wherein the composition further comprises electrolytes.

Embodiment 33. The method of embodiment 32, wherein the electrolytes comprise sodium, potassium, calcium, magnesium, chloride, bicarbonate, phosphate, sulfate, or a combination of two or more thereof.

Embodiment 34. The method of embodiment 32, wherein the electrolytes comprise sodium sulfate, sodium bicarbonate, sodium chloride, potassium chloride, or a combination of two or more thereof.

Embodiment 35. The method of any one of embodiments 30 to 32, wherein the composition comprises from about 0.05 wt % to about 20 wt % of electrolytes.

Embodiment 36. The method of any one of embodiments 32 to 34, wherein the composition comprises from about 0.05 wt % to about 5 wt % of electrolytes.

Embodiment 37. The method of any one of embodiments 32 to 34, wherein the composition comprises from about 0.1 wt % to about 2 wt % of electrolytes.

Embodiment 38. The method of embodiment 13, wherein the composition comprises about 1.1 wt % of tranexamic acid, about 4.6 wt % of polyethylene glycol having an average molecular weight of about 3,350, about 4.0 wt % of glucose, about 0.57 wt % of sodium sulfate, about 0.17 wt % of sodium bicarbonate, about 0.15 wt % of sodium chloride, and about 0.07 wt % of potassium chloride.

Embodiment 39. The method of embodiment 13, wherein the composition comprises about 1.1 wt % of tranexamic acid, about 4.2 wt % of polyethylene glycol having an average molecular weight of about 3,350, about 5.0 wt % of glucose, about 0.4 wt % of sodium sulfate, about 0.12 wt % of sodium bicarbonate, about 0.1 wt % of sodium chloride, and about 0.05 wt % of potassium chloride.

Embodiment 40. The method of embodiment 13, wherein the composition comprises about 0.8 wt % of tranexamic acid, about 4.2 wt % of polyethylene glycol having an average molecular weight of about 3,350, about 5.0 wt % of glucose, about 0.57 wt % of sodium sulfate, about 0.17 wt % of sodium bicarbonate, about 0.15 wt % of sodium chloride, and about 0.07 wt % of potassium chloride.

Embodiment 41. The method of embodiment 13, wherein the pharmaceutical composition comprises about 0.5 wt % to about 2 wt % of tranexamic acid, about 2 wt % to about 6 wt % of polyethylene glycol, about 2 wt % to about 6 wt % of glucose, and about 0.1 wt % to about 2 wt % of at least one electrolyte.

Embodiment 42. The method of embodiment 11, wherein the pharmaceutical composition comprises about 1 wt % to about 1.2 wt % of tranexamic acid, about 4 wt % to about 4.8 wt % of polyethylene glycol, about 3.5 wt % to about 5.5 wt % of glucose, and about 0.5 wt % to about 1.1 wt % of at least one electrolyte.

Embodiment 43. The method of embodiment 40 or 41, wherein the polyethylene glycol has an average molecular weight from about 100 Daltons to about 30,000 Daltons.

Embodiment 44. The method of embodiment 40 or 41, wherein the polyethylene glycol has an average molecular weight from about 1,000 Daltons to about 10,000 Daltons.

Embodiment 45. The method of embodiment 40 or 41, wherein the polyethylene glycol has an average molecular weight of about 3,500 Daltons.

Embodiment 46. The method of embodiment 40 or 41, wherein the electrolytes comprise sodium, potassium, calcium, magnesium, chloride, bicarbonate, phosphate, sulfate, or a combination of two or more thereof.

Embodiment 47. The method of embodiment 40 or 41, wherein the electrolytes comprise sodium sulfate, sodium bicarbonate, sodium chloride, potassium chloride, or a combination of two or more thereof.

Embodiment 48. A method for lowering blood glucose levels in a diabetic patient in need thereof, the method comprising orally administering to the diabetic patient a therapeutically effective amount of an aqueous pharmaceutical composition prior to a surgical procedure to lower blood glucose levels; wherein the composition comprises a polyethylene glycol.

Embodiment 49. A method for lowering blood glucose levels in a diabetic patient in need thereof, the method comprising orally administering to the diabetic patient a therapeutically effective amount of an aqueous pharmaceutical composition after a surgical procedure to lower blood glucose levels; wherein the composition comprises a polyethylene glycol.

Embodiment 50. A method for lowering blood glucose levels in a diabetic patient in need thereof, the method comprising orally administering to the diabetic patient a therapeutically effective amount of an aqueous pharmaceutical composition prior to a surgical procedure and after a surgical procedure to lower blood glucose levels; wherein the composition comprises a polyethylene glycol.

Embodiment 51. The method of any one of embodiments 48 or 50, further comprising administering the composition during the surgical procedure.

Embodiment 52. The method of any one of embodiments 48 to 50, wherein the method for lowering blood glucose levels is a method for reducing post-surgical blood glucose levels.

Embodiment 53. The method of any one of embodiments 49 to 52, comprising administering the composition for at least two days after the surgical procedure.

Embodiment 54. The method of any one of embodiments 49 to 52, comprising administering the composition for at least three days after the surgical procedure.

Embodiment 55. The method of any one of embodiments 49 to 52, comprising administering the composition from one to four days after the surgical procedure.

Embodiment 56. The method of any one of embodiments 49 to 52, comprising administering the composition from one to ten days after the surgical procedure.

Embodiment 57. The method of any one of embodiments 48 to 56, wherein the polyethylene glycol has an average molecular weight from about 100 Daltons to about 50,000 Daltons.

Embodiment 58. The method of any one of embodiments 48 to 56, wherein the polyethylene glycol has an average molecular weight from about 100 Daltons to about 30,000 Daltons.

Embodiment 59. The method of any one of embodiments 48 to 56, wherein the polyethylene glycol has an average molecular weight from about 1,000 Daltons to about 10,000 Daltons.

Embodiment 60. The method of any one of embodiments 48 to 56, wherein the polyethylene glycol has an average molecular weight of about 3,500 Daltons.

Embodiment 61. The method of any one of embodiments 48 to 60, wherein the composition comprises from about 1 wt % to about 25 wt % of polyethylene glycol.

Embodiment 62. The method of any one of embodiments 48 to 61, wherein the composition comprises from about 1 wt % to about 15 wt % of polyethylene glycol.

Embodiment 63. The method of any one of embodiments 48 to 61, wherein the composition comprises from about 1 wt % to about 10 wt % of polyethylene glycol.

Embodiment 64. The method of any one of embodiments 48 to 61, wherein the composition comprises from about 2 wt % to about 6 wt % of polyethylene glycol.

Embodiment 65. The method of any one of embodiments 48 to 64, wherein the composition further comprises glucose.

Embodiment 66. The method of embodiment 65, wherein the composition comprises from about 1 wt % to about 25 wt % of glucose.

Embodiment 67. The method of embodiment 65, wherein the composition comprises from about 1 wt % to about 15 wt % of glucose.

Embodiment 68. The method of embodiment 65, wherein the composition comprises from about 1 wt % to about 10 wt % of glucose.

Embodiment 69. The method of embodiment 65, wherein the composition comprises from about 2 wt % to about 6 wt % of glucose.

Embodiment 70. The method of any one of embodiments 48 to 69, wherein the composition further comprises electrolytes.

Embodiment 71. The method of embodiment 70, wherein the electrolytes comprise sodium, potassium, calcium, magnesium, chloride, bicarbonate, phosphate, sulfate, or a combination of two or more thereof.

Embodiment 72. The method of embodiments 70, wherein the electrolytes comprise sodium sulfate, sodium bicarbonate, sodium chloride, potassium chloride, or a combination of two or more thereof.

Embodiment 73. The method of any one of embodiments 70 to 72, wherein the composition comprises from about 0.05 wt % to about 20 wt % of electrolytes.

Embodiment 74. The method of any one of embodiments 70 to 72, wherein the composition comprises from about 0.05 wt % to about 5 wt % of electrolytes.

Embodiment 75. The method of any one of embodiments 70 to 72, wherein the composition comprises from about 0.1 wt % to about 2 wt % of electrolytes.

Embodiment 76. An aqueous composition comprising about 1.1 wt % of tranexamic acid, about 4.2 wt % of polyethylene glycol having an average molecular weight of about 3,350, and about 5.0 wt % of glucose.

Embodiment 77. The composition of embodiment 76, further comprising about 0.4 wt % of sodium sulfate, about 0.12 wt % of sodium bicarbonate, about 0.1 wt % of sodium chloride, and about 0.05 wt % of potassium chloride.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

The following examples are for purposes of illustration and are not intended to limit the spirit or scope of the disclosure or claims.

### Example 1

Seventy-six patients undergoing coronary artery bypass graft (CABG) surgery and/or heart valve replacement surgery requiring cardiopulmonary bypass (CBP) were randomized in a double-blind, parallel, placebo-controlled Phase 2 clinical trial of Formulation B. Patients received Formulation B or placebo (vehicle alone) immediately after surgery via nasogastric/orogastric tube where Formulation B was administered by drip through the tube over a period of 1 to 24 hours, depending on the patient. Formulation B was administered in this manner for 4 days post-surgery (where administration of Formulation B on the day of, but after surgery, was day 0).

The primary objective was to determine if enteral administration of Formulation B improved one or more of the following post-operative outcomes: mortality rate; length of intensive care unit stay, length of hospital stay, and return of normal gut function. The secondary objectives were analyzed 30 days after the patient had surgery, and included resolution of organ dysfunction (pulmonary, renal, hepatic, central nervous system), and inflammatory response measured by SIRS criteria.

Analysis of the data unexpectedly and surprisingly revealed that in the small subset of subject with diabetes, treatment with Formulation B had an unexpected decrease in serum glucose levels following surgery when compared to diabetic patients receiving vehicle.

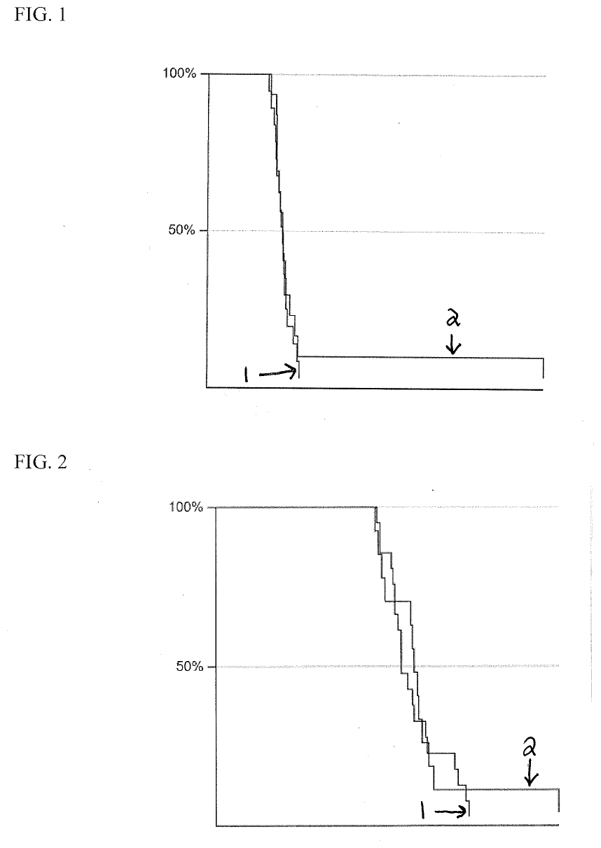

For non-diabetic patients administered Formulation B and vehicle, FIGS. 1 and 2 show that there was no statistically difference in serum glucose levels prior to administration of Formula B (as shown in FIG. 1), or after administration of four doses of Formulation B (as shown in FIG. 2). The time course of blood glucose levels over all 4 days in the Formulation B and vehicle groups is shown in FIG. 9.

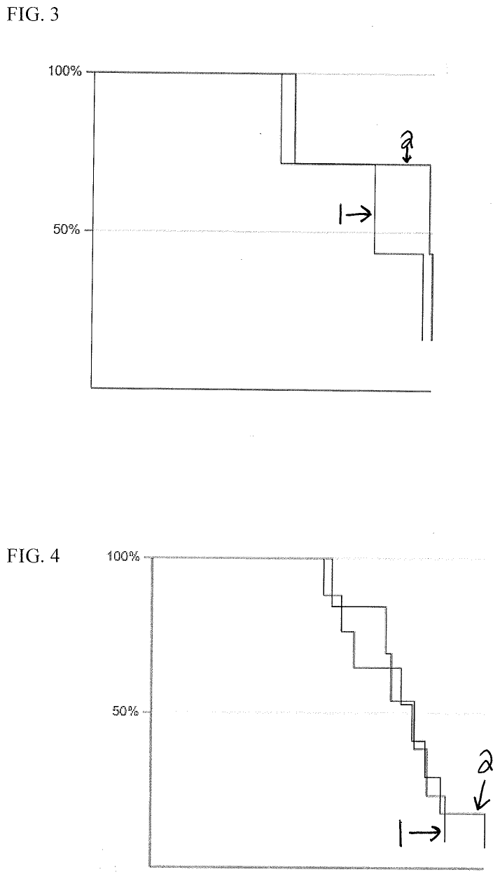

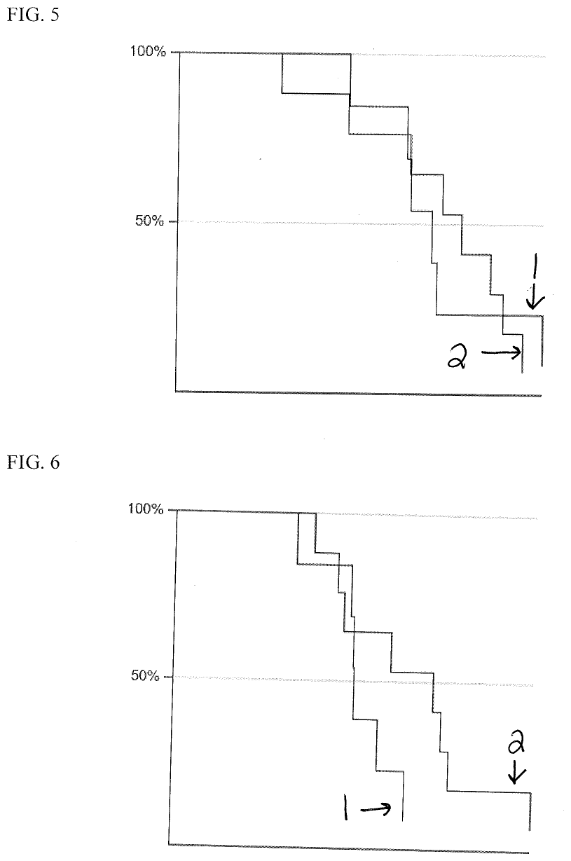

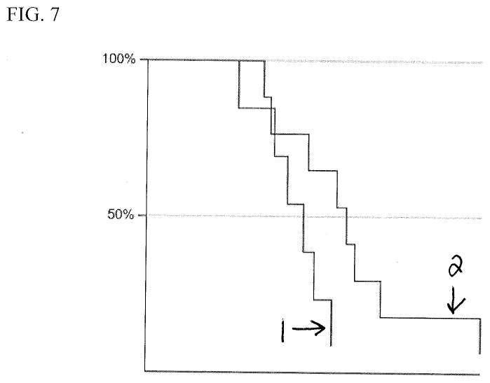

For diabetic patients administered Formulation B and vehicle, FIGS. 3-7 show the trend in improvement in glucose function over the course of 4 days, where diabetic patients administered Formulation B had progressively lower glucose levels each day. At Day 4 (FIG. 7), the difference in serum glucose levels for patients treated with Formulation B or vehicle alone was statistically significant. As noted herein, day 0 is the day of surgery when the patient was first administered Formulation B or vehicle.

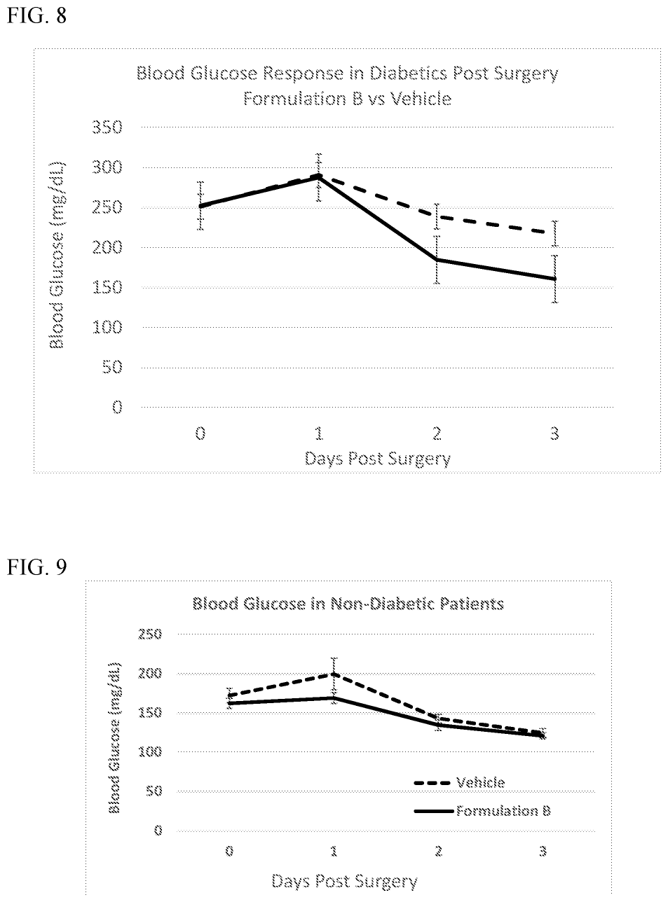

As shown in FIG. 8, diabetic subjects treated with Formulation B show a trending reduction in glucose levels after surgery, compared with vehicle, including a significant decrease at 3 days after surgery (p =.04).

### Example 2

In this prophetic example, a clinical trial is conducted for patients undergoing bowel resections. The patients are administered Formulation A or placebo the day before surgery. After the bowel resection, the patients are administered Formulation A the same day, but after, the surgical procedure, and for at least 3 days thereafter. Diabetic patients administered Formulation A show statistically significantly lower blood glucose levels within 1 to 3 days after surgery when compared to diabetic patients who are administered placebo, thus replicating the results shown in Example 1.

Without intending to be bound by any theory of the invention, tranexamic acid prevents plasmin (antiplasmin) from binding to and degrading fibrin and preserves the framework of fibrin's matrix structure. The anti-proteolytic effects of tranexamic acid work in three ways. At the bowel level they reduce and prevent protease migration intravascularly. Tranexamic acid preserves and protects some insulin receptors but also eliminates intravascular proteases and allows anabolic reformation of the receptors. The pharmaceutical compositions described herein are particularly effective for diabetic patients because they have Type II diabetes, which is characterized by high blood sugar, insulin resistance, and relative lack of insulin. They are already very dependent on many receptors producing any cell signaling that they can to try to promote the absorption of glucose from the blood (especially into fat, live and skeletal muscles), promote glycogenesis and lipogenesis. A non-diabetic person can handle some loss of the number of insulin receptors since there is normal redundancy but also because they are not resistant to insulin. However, a Type II diabetic is teetering on the edge, and any loss of insulin receptor signally immediately starts to throw their sugars off and cause hyperglycemia.

It is understood that the examples and embodiments described herein are for illustrative purposes only and that various modifications or changes in light thereof will be suggested to persons skilled in the art and are to be included within the spirit and purview of this application and scope of the appended claims.

What is claimed is:

1. A pharmaceutical composition comprising a protease inhibitor for use in a method of lowering blood glucose levels in a diabetic patient.

2. The composition for use as in claim 1, wherein the composition is orally administered as a liquid dosage form.

3. The composition for use as in claim 1, wherein the composition is orally administered as a solid dosage form.

4. The composition for use as in any one of claims 1 to 3, wherein the diabetic patient is undergoing a surgical procedure to lower blood glucose levels.

5. The composition for use as in claim 4, wherein the composition is orally administered to the patient prior to the surgical procedure.

6. The composition for use as in claim 4, wherein the composition is orally administered to the patient after the surgical procedure.

7. The composition for use as in claim 4, wherein the composition is orally administered to the patient prior to and after the surgical procedure.

8. The composition for use as in any one of claims 4 to 7 wherein the composition is orally administered to the patient during the surgical procedure.

9. The composition for use as in any one of claims 4 to 8, wherein the method for lowering blood glucose levels is a method for reducing post-surgical blood glucose levels.

10. The composition for use as in any one of claim 6 or 9, wherein the composition is administered for at least two days after the surgical procedure.

11. The composition for use as in any one of claim 6 or 9, wherein the composition is administered for at least three days after the surgical procedure.

12. The composition for use as in any one of claim 6 or 9, wherein the composition is administered from one to four days after the surgical procedure.

13. The composition for use as in any one of claim 6 or 9, wherein the composition is administered from one to ten days after the surgical procedure.

14. The composition for use as in any one of claims 1 to 3, wherein the diabetic patient is not undergoing a surgical procedure or is in a non-hospital setting.

15. The composition for use as in claim 14, wherein the composition is administered chronically.

16. The composition for use as in any one of claims 1 to 15, wherein the protease inhibitor is a serine protease inhibitor, a matrix metalloproteinase inhibitor, or a lipase inhibitor.

17. The composition for use as in any one of claims 1 to 15, wherein the protease inhibitor is a serine protease inhibitor.

18. The composition for use as in any one of claims 1 to 15, wherein the protease inhibitor is tranexamic acid, gabexate mesilate, camostat mesilate, nafamostat mesilate, α-1-anti-trypsin, α-2-macroglobulin, pancreatic lipase, gastric lipase, pancreatic lipase-related protein 1, pancreatic lipase-related protein 2, aprotinin, or a combination of two or more thereof.

19. The composition for use as in any one of claims 1 to 15, wherein the protease inhibitor is tranexamic acid.

20. The composition for use as in any one of claims 1 to 19, wherein the composition comprises from about 0.1 wt % to about 25 wt % of the protease inhibitor.

21. The composition for use as in any one of claims 1 to 19, wherein the composition comprises from about 0.1 wt % to about 10 wt % of the protease inhibitor.

22. The composition for use as in any one of claims 1 to 19, wherein the composition comprises from about 0.1 wt % to about 5 wt % of the protease inhibitor.

23. The composition for use as in any one of claims 1 to 19, wherein the composition comprises from about 0.1 wt % to about 2 wt % of the protease inhibitor.

24. The composition for use as in any of claims 1 to 23, wherein the composition further comprises polyethylene glycol.

25. The composition for use as in claim 24, wherein the polyethylene glycol has an average molecular weight from about 100 Daltons to about 50,000 Daltons.

26. The composition for use as in claim 24, wherein the polyethylene glycol has an average molecular weight from about 100 Daltons to about 30,000 Daltons.

27. The composition for use as in claim 24, wherein the polyethylene glycol has an average molecular weight from about 1,000 Daltons to about 10,000 Daltons.

28. The composition for use as in claim 24, wherein the polyethylene glycol has an average molecular weight of about 3,500 Daltons.

29. The composition for use as in any one of claims 24-28, wherein the composition comprises from about 1 wt % to about 25 wt % of polyethylene glycol.

30. The composition for use as in any one of claims 24-28, wherein the composition comprises from about 1 wt % to about 15 wt % of polyethylene glycol.

31. The composition for use as in any one of claims 24-28, wherein the composition comprises from about 1 wt % to about 10 wt % of polyethylene glycol.

32. The composition for use as in any one of claims 24-28, wherein the composition comprises from about 2 wt % to about 6 wt % of polyethylene glycol.

33. The composition for use as in any one of claims 1-32, wherein the composition further comprises glucose.

34. The composition for use as in claim 33, wherein the composition comprises from about 1 wt % to about 25 wt % of glucose.

35. The composition for use as in claim 33, wherein the composition comprises from about 1 wt % to about 15 wt % of glucose.

36. The composition for use as in claim 33, wherein the composition comprises from about 1 wt % to about 10 wt % of glucose.

37. The composition for use as in claim 33, wherein the composition comprises from about 2 wt % to about 6 wt % of glucose.

38. The composition for use as in any one of claims 1 to 37, wherein the composition further comprises electrolytes.

39. The composition for use as in claim 38, wherein the electrolytes comprise sodium, potassium, calcium, magnesium, chloride, bicarbonate, phosphate, sulfate, or a combination of two or more thereof

40. The composition for use as in claim 38, wherein the electrolytes comprise sodium sulfate, sodium bicarbonate, sodium chloride, potassium chloride, or a combination of two or more thereof.

41. The composition for use as in any one of claims 38 to 40, wherein the composition comprises from about 0.05 wt % to about 20 wt % of electrolytes.

42. The composition for use as in any one of claims 38 to 40, wherein the composition comprises from about 0.05 wt % to about 5 wt % of electrolytes.

43. The composition for use as in any one of claims 38 to 40, wherein the composition comprises from about 0.1 wt % to about 2 wt % of electrolytes.

44. The composition for use as in claim 19, wherein the composition comprises about 1.1 wt % of tranexamic acid, about 4.6 wt % of polyethylene glycol having an average molecule weight of about 3,350, about 4.0 wt % of glucose, about 0.57% of sodium sulfate, about 0.17 wt % of sodium bicarbonate, about 0.15% wt % of sodium chloride, and about 0.07 wt % of potassium chloride.

45. The composition for use as in claim 19, wherein the composition comprises about 1.1 wt % of tranexamic acid, about 4.2 wt % of polyethylene glycol having an average molecule weight of about 3,350, about 5.0 wt % of glucose, about 0.4 wt % of sodium sulfate, about 0.12 wt % of sodium bicarbonate, about 0.1% wt % of sodium chloride, and about 0.05 wt % of potassium chloride.

46. The composition for use as in claim 19, wherein the composition comprises about 0.8 wt % of tranexamic acid, about 4.2 wt % of polyethylene glycol having an average molecule weight of about 3,350, about 5.0 wt % of glucose, about 0.57 wt % of sodium sulfate, about 0.17 wt % of sodium bicarbonate, about 0.15% wt % of sodium chloride, and about 0.07 wt % of potassium chloride.

47. The composition for use as in claim 19, wherein the composition comprises about 0.5 wt % to about 2 wt % of tranexamic acid, about 2 wt % to about 6 wt % of polyethylene glycol, about 2 wt % to about 6 wt % of glucose, and about 0.1 wt % to about 2 wt % of at least one electrolyte.

48. The composition for use as in claim 19, wherein the pharmaceutical composition comprises about 1 wt % to about 1.2 wt % of tranexamic acid, about 4 wt % to about 4.8 wt % of polyethylene glycol, about 3.5 wt % to about 5.5 wt % of glucose, and about 0.5 wt % to about 1.1 wt % of at least one electrolyte.

49. The composition for use as in claim 47 or 48, wherein the polyethylene glycol has an average molecular weight from about 100 Daltons to about 30,000 Daltons.

50. The composition for use as in claim 47 or 48, wherein the polyethylene glycol has an average molecular weight from about 1,00 Daltons to about 10,000 Daltons.

51. The composition for use as in claim 47 or 48, wherein the polyethylene glycol has an average molecular weight of about 3,500 Daltons.

52. The composition for use as in claim 47 or 48, wherein the electrolytes comprise sodium, potassium, calcium, magnesium, chloride, bicarbonate, phosphate, sulfate, or a combination of two or more thereof.

53. The composition for use as in claim 47 or 48, wherein the electrolytes comprise sodium sulfate, sodium bicarbonate, sodium chloride, potassium chloride, or a combination of two or more thereof.

54. The composition for use as in any one the preceding claims, wherein the composition preserves and protects insulin receptors.

55. A pharmaceutical composition comprising a polyethylene glycol for use in a method for lowering blood glucose levels in a diabetic patient.

56. The composition for use as in claim 55, wherein the composition is orally administered as a liquid dosage form.

57. The composition for use as in claim 56, wherein the composition is orally administered as a solid dosage form.

58. The composition for use as in claim 55 or 57, wherein the diabetic patient is undergoing a surgical procedure to lower blood glucose levels.

59. The composition for use as in claim 58, wherein the composition is orally administered to the patient prior to the surgical procedure.

60. The composition for use as in claim 58, wherein the composition is orally administered to the patient after the surgical procedure.

61. The composition for use as in claim 58, wherein the composition is orally administered to the patient prior to and after the surgical procedure.

62. The composition for use as in any one of claims 58 to 61, wherein the composition is orally administered to the patient during the surgical procedure.

63. The composition for use as in any one of claims 58 to 61, wherein the method for lowering blood glucose levels is a method for reducing post-surgical blood glucose levels.

64. The composition for use as in any one of claim 60 or 63, wherein the composition is administered for at least two days after the surgical procedure.

65. The composition for use as in any one of claim 60 or 63, wherein the composition is administered for at least three days after the surgical procedure.

66. The composition for use as in any one of claim 60 or 63, wherein the composition is administered from one to four days after the surgical procedure.

67. The composition for use as in any one of claim 60 or 63, comprising administering the composition from one to ten days after the surgical procedure.

68. The composition for use as in any one of claims 55 to 57, wherein the diabetic patient is not undergoing a surgical procedure or is in a non-hospital setting.

69. The composition for use as in claim 68, wherein the composition is administered chronically.

70. The composition for use as in any one of claims 55 to 69, wherein the polyethylene glycol has an average molecular weight from about 100 Daltons to about 50,000 Daltons.

71. The composition for use as in any one of claims 55 to 69, wherein the polyethylene glycol has an average molecular weight from about 100 Daltons to about 30,000 Daltons.

72. The composition for use as in any one of claims 55 to 69, wherein the polyethylene glycol has an average molecular weight from about 1,000 Daltons to about 10,000 Daltons.

73. The composition for use as in any one of claims 55 to 69, wherein the polyethylene glycol has an average molecular weight of about 3,500 Daltons.

74. The composition for use as in any one of claims 55 to 69, wherein the composition comprises from about 1 wt % to about 25 wt % of polyethylene glycol.

75. The composition for use as in any one of claims 55 to 69, wherein the composition comprises from about 1 wt % to about 15 wt % of polyethylene glycol.

76. The composition for use as in any one of claims 55 to 69, wherein the composition comprises from about 1 wt % to about 10 wt % of polyethylene glycol.

77. The composition for use as in any one of claims 55 to 69, wherein the composition comprises from about 2 wt % to about 6 wt % of polyethylene glycol.

78. The composition for use as in any one of claims 55 to 77, wherein the composition further comprises glucose.

79. The composition for use as in claim 78, wherein the composition comprises from about 1 wt % to about 25 wt % of glucose.

80. The composition for use as in claim 78, wherein the composition comprises from about 1 wt % to about 15 wt % of glucose.

81. The composition for use as in claim 78, wherein the composition comprises from about 1 wt % to about 10 wt % of glucose.

82. The composition for use as in claim 78, wherein the composition comprises from about 2 wt % to about 6 wt % of glucose.

83. The composition for use as in any one of claims 55 to 82, wherein the composition further comprises electrolytes.

84. The composition for use as in claim 83, wherein the electrolytes comprise sodium, potassium, calcium, magnesium, chloride, bicarbonate, phosphate, sulfate, or a combination of two or more thereof.

85. The composition for use as in claim 83, wherein the electrolytes comprise sodium sulfate, sodium bicarbonate, sodium chloride, potassium chloride, or a combination of two or more thereof.

86. The composition for use as in any one of claims 83 to 85, wherein the composition comprises from about 0.05 wt % to about 20 wt % of electrolytes.

87. The composition for use as in any one of claims 83 to 85, wherein the composition comprises from about 0.05 wt % to about 5 wt % of electrolytes.

88. The composition for use as in any one of claims 83 to 85, wherein the composition comprises from about 0.1 wt % to about 2 wt % of electrolytes.

89. The composition for use as in any one of claims 55-88, wherein the composition preserves and protects insulin receptors.

90. An aqueous composition comprising about 1.1 wt % of tranexamic acid, about 4.2 wt % of polyethylene glycol having an average molecule weight of about 3,350, and about 5.0 wt % of glucose.

91. The composition of claim 90, further comprising about 0.4 wt % of sodium sulfate, about 0.12 wt % of sodium bicarbonate, about 0.1 wt % of sodium chloride, and about 0.05 wt % of potassium chloride.

92. A composition, as in the any of the preceding claims, for use in a method of treating diabetes.

93. A composition, as in any of the preceding claims, for use in a method of lowering blood glucose levels in a diabetic patient.

94. A composition, as in any of the preceding claims, for use in a method of improving glycemic control in a diabetic patient.
